(19) United States
(12) Patent Application Publication
(10) Pub. No.: US 2016/0045158 A1
(43) Pub. Date: Feb. 18, 2016

(54) DEVICES, SYSTEMS, AND METHODS FOR TRANSDERMAL DELIVERY OF COMPOUNDS

(71) Applicant: PROMETHEON PHARMA, LLC, Gainesville, FL (US)

(72) Inventor: Stephen I. HSU, Gainesville, FL (US)

(21) Appl. No.: 14/776,596

(22) PCT Filed: Mar. 17, 2014
(86) PCT No.: PCT/US2014/030259

§ 371 Date: Sep. 14, 2015

Related U.S. Application Data
(60) Provisional application No. 61/800,378, filed on Mar. 15, 2013.
(60) Provisional application No. 61/802,213, filed on Mar. 15, 2013.

(51) Int. Cl. A61B 5/00; A61B 5/145; A61B 5/1486

(52) U.S. Cl. CPC A61B 5/4839; A61B 5/4848; A61B 5/14865; A61B 5/14532; A61B 5/6833; A61B 2017/0023

(57) ABSTRACT

A device for measurement and monitoring of a subject simultaneously with transdermal or transmucosal delivery of a therapeutic agent at a contact site with the subject's skin includes a transdermal sensor adapted and configured to detect a specific indicator that is either the therapeutic agent itself or a biomarker that is affected by the therapeutic agent, a therapeutic-agent-containing formulation for passive or active transdermal drug delivery, wherein the formulation includes a dermoadhesive agent to adhere the underside of the sensor housing unit to the skin, and a separate circumferential self-adhesive patch can be adapted and configured to hold the sensor and its housing unit firmly to the skin at the contact site for multiple days.

### CROSS-REFERENCE TO RELATED APPLICATIONS

This application claims the benefit of and priority to U.S. Provisional Patent Application No. 61/800,378, filed Mar. 15, 2013, and U.S. Provisional Patent Application No. 61/802,213, filed Mar. 15, 2013, the entire contents of each being incorporated by reference herein.

### BACKGROUND

The transdermal patch segment of the pharmaceutical industry currently commands a relatively small share ($3 billion in 2010) of the rapidly growing global drug delivery market ($21.5 billion in 2010, projected growth to $31.5 billion by 2015). Approved patches are currently available for drugs with properties that lend themselves to passive permeation across the skin when applied topically such as estrogen, nicotine, nitroglycerin, scopolamine, fentanyl and clonidine.

To date, patch-based penetration enhancement formulations have been limited to delivering small chemical drugs below a molecular weight size of 500 daltons (the so-called “rule of 500”) due to the physical constraints of effectively transporting large water-soluble compounds across the thick, keratin-rich, armor-like outer layer of skin (stratum corneum). The underlying cellular layers that comprise the viable epidermis, also present a rate-limiting barrier to transdermal drug delivery after successful penetration of the stratum corneum. The barrier to drug diffusion across the epidermis is probably the presence of tight junctions. Removal of the full epidermis increased skin permeability by 1-2 orders of magnitude depending on the molecule delivered.

### SUMMARY

A device for measurement and monitoring of a subject simultaneously with transdermal or transmucosal delivery of a therapeutic agent at a contact site with the subject's skin can include a transdermal sensor adapted and configured to detect a specific indicator that is either the therapeutic agent itself or a biomarker that is affected by the therapeutic agent, a therapeutic-agent-containing formulation for passive or active transdermal drug delivery, wherein the formulation includes a dermoadhesive agent to adhere the underside of the sensor housing unit to the skin.

In at least some embodiments, a separate circumferential self-adhesive patch can be adapted and configured to hold the sensor and its housing unit firmly to the skin at the contact site for multiple days.

The therapeutic agent can be any suitable compound or composition such as, but not limited to, small molecule drugs, large molecule drugs, therapeutic peptides, DNA, and microRNA, combinations thereof, or the like. In some embodiments, the therapeutic agent can be human native insulin and insulin analogues such as insulin aspart or insulin glargine.

The transdermal sensor can include one or more electrodes adapted and configured for insertion into the subepidermal interstitial space. In some embodiments, the transdermal sensor can be disposable and biodegradable. The transdermal sensor can be an enzymatic electrochemical sensor.

In some embodiments, the transdermal sensor includes one or more electrodes adapted to pierce the skin. Immobilized glucose oxidase can be coupled to at least one of the electrodes. The immobilized glucode oxidase can catalyze oxidation of glucose to hydrogen peroxide and D-glucono-δ-lactone and the hydrogen peroxide can react with a surface of the electrode to produce electric current. In some embodiments, the specific indicator can be an interstitial glucose level that is correlated with a blood glucose level of the subject.

In some embodiments, the transdermal sensor can be adapted and configured to continuously detect the specific indicators. The transdermal sensor can be adapted and configured to detect the specific indicator at an interval T, wherein T is selected from the group consisting of: less than about 1 second, between about 1 second and about 5 seconds, between about 5 seconds and about 10 seconds, between about 10 seconds and about 30 seconds, between about 30 seconds and about 60 seconds, between about 1 minute and about 5 minutes, between about 5 minutes and about 10 minutes, between about 10 minutes and about 15 minutes, between about 15 minutes and about 30 minutes, and between about 30 minutes and about 60 minutes.

A surface of the device adjacent to the contact site can be between about 1 and about 25 square centimeters in size. In some embodiments, the device can be between about 1 and about 25 cubic centimeters in size.

In some embodiments, measurement of the specific indicator and delivery of the therapeutic agent both can occur at the contact site.

The dermoadhesive agent in the therapeutic-agent-containing formulation can be selected from the group including of propylene glycol, dipropylene glycol, polyethylene glycol, glycerine, butylene glycol, glycol derivatives with glycerol esters, and non-ionizable glycol ether derivatives.

The storage reservoir can be a transdermal skin patch. The therapeutic-agent-containing formulation can include a thermo-sensitive polymer. In some embodiments, the thermo-sensitive polymer can include a poloxamer or a poloxamine. The poloxamer can be poloxamer 188.

The storage reservoir can release the therapeutic agent via passive or active diffusion across a skin surface that is not being physically disrupted with technologies selected from the group including but not limited to thermal or laser ablation, microneedles, high pressure jet injection, ultrasound, sonar and iontophoresis.

In some embodiments, the device can further include a transmitter communicatively coupled with the sensor, the transmitter adapted and configured to transmit data generated by the sensor. The transdermal sensor can include circuitry that interfaces with the transmitter.

The transmitter can be a wireless transmitter. The transmitter can transmit data at a frequency between about 402 MHz and about 433 MHz. In some embodiments, the transmitter transmits data at a frequency between about 2,400 MHz and about 2,480 MHz. In some embodiments, the frequency is unique to each transmitter and receiver.

The device can further include a receiver adapted and configured to communicate with the transmitter. The receiver can be adapted and configured to store data received from the transmitter in memory.

The receiver can be adapted and configured to transmit stored data received from the transmitter to another computing device that utilizes a software program to graphically display data in a visual format that allows interpretation by the subject or a health care provider to make clinical decisions such as adjustment of the therapeutic dose delivered. The computing device can store the data generated by the software.

In at least one aspect of this disclosure, a device includes a transdermal sensor adapted and configured to detect a specific indicator that is either the therapeutic agent itself or a biomarker that is affected by the therapeutic agent, a surface commonly referred to as a film or laminate or backing that is coated with a therapeutic-agent-containing formulation that serves as a storage reservoir for passive or active transdermal drug delivery, and a dermoadhesive agent present in the formulation adapted and configured to adhere the underside of the sensor housing unit to the skin and provide transdermal delivery of a therapeutic agent at a contact site.

A separate circumferential self-adhesive patch can be included that is adapted and configured to hold the sensor and its housing unit and the storage reservoir firmly to the skin at the contact site for multiple days.

The device can further include a transmitter communicatively coupled with the sensor, the transmitter adapted and configured to transmit data generated by the sensor.

In at least one aspect of this disclosure, a method of monitoring a subject includes applying a device of claim 1 to the skin of a subject. The device can be maintained in place on the skin of the subject for at least 1 day. The device can be maintained in place on the skin of the subject for at least 7 days. The device can be maintained in place on the skin of the subject for at least 14 days.

The method can further include transmitting instructions to modulate a dosage of the therapeutic agent to the subject based on the specific indicator detected by the transdermal sensor.

The storage reservoir can release the therapeutic agent via passive or active diffusion across a skin surface that has not been physical disrupted with technologies selected from but not limited to the group consisting of: thermal or laser ablation, microneedles, high pressure jet injection, ultrasound, sonar and iontophoresis.

In at least one aspect of the present disclosure, a method includes coating the contact surface of the transdermal sensor system with the therapeutic-agent-containing formulation that constitutes the storage reservoir.

In at least one aspect of the present disclosure, a method of making the device includes coating the contact surface of the transdermal sensor system with the therapeutic-agent-containing formulation that constitutes the storage reservoir. In at least one aspect of the present disclosure, a method of making the device includes coupling the transdermal sensor system to a transmitter.

In at least one aspect of the present disclosure, a modulatable transdermal patch can include a perforated substrate including a plurality of segments defined by one or more perforations, and a therapeutic-agent-containing formulation applied across at least a portion of one side of the perforated substrate, such that the therapeutic-agent-containing formulation is distributed across the plurality of segments. The plurality of perforations define between 4 and 10 segments.

In at least one aspect of the present disclosure, a kit includes a device as disclosed herein and instructions for use.

In at least one aspect of the present disclosure, a kit includes a transdermal sensor adapted and configured to detect a specific indicator that is either the therapeutic agent itself or a biomarker that is affected by the therapeutic agent, a storage reservoir suitable for storing a therapeutic-agent-containing formulation for passive or active transdermal drug delivery, a dermoadhesive agent present in the formulation adapted and configured to hold the sensor and its housing unit to the skin, a circumferential self-adhesive patch adapted and configured to hold the sensor and its housing unit and the storage reservoir firmly to the skin at the contact site for multiple days, and instructions for use.

In at least one aspect of the present disclosure, a kit includes a transdermal sensor adapted and configured to detect a specific indicator that is either the therapeutic agent itself or a biomarker that is affected by the therapeutic agent, a therapeutic-agent-containing formulation for passive or active transdermal drug delivery, and a dermoadhesive agent present in the formulation adapted and configured to hold the sensor and its housing unit to the skin. A circumferential self-adhesive patch adapted and configured to hold the sensor and its housing unit and the storage reservoir firmly to the skin and provide transdermal delivery of a therapeutic agent at the contact site, and instructions for use.

### BRIEF DESCRIPTION OF THE DRAWINGS

FIG. 1 depicts a device 100 that allows for continuous ambulatory measurement and monitoring of a subject simultaneously with transdermal delivery of a therapeutic agent at a direct contact site 102 (bio-interface with stratum corneum) on the subject's skin 104 (depicts full thickness skin comprised of stratum corneum, epidermis and dermis). The sensor 114 extends through the skin 104 into the underlying subcutaneous interstitial space.

FIG. 1A depicts a similar device to that of FIG. 1 except the dermoadhesive does not form a separate layer and is included in the formulation of the drug formulation storage reservoir 108.

FIG. 2 depicts a method 400 of utilizing a combined delivery and monitoring device.

FIGS. 3A-3C depicts modulatable transdermal patches that can allow a subject to adjust the dosing of a therapeutic agent by removing (before applying the patch) one or more pre-perforated segments of the patch liner (or optionally the entire liner to receive full dose) to expose a greater or lesser surface area for drug delivery.

FIGS. 3AA-3AC depicts modulatable transdermal patches that can allow a subject to adjust the dosing of a therapeutic agent by removing (before applying the patch) one or more pre-perforated segments of the patch liner (or optionally the entire liner to receive full dose) to expose a greater or lesser surface area for drug delivery.

FIG. 4 depicts differential scanning calorimetry curves indicating the thermal properties of biopolymer formulations of 1 mM *Br1 in differing proportions of polaxamer 188 (P188) and propylene glycol (PG) and with the addition of 0.4 M laurocapram (LP).

FIG. 5 shows a chart of experimentally determined glargine flux in a full thickness human skin model.

FIG. 6 shows a chart of experimentally determined detemir flux in a full thickness human skin model.

FIG. 7 shows a chart of experimentally determined insulin lispro flux in a full thickness human skin model.

FIG. 8 shows a chart of experimentally determined insulin glulisine flux in a full thickness human skin model.

FIG. 9 shows a chart of experimentally determined insulin aspart flux in a full thickness human skin model.

FIG. 10 shows a chart of experimentally determined regular insulin flux in a full thickness human skin model.

FIG. 11 shows the localization at the dermal-epidermal junction of glargine microprecipitates formed after the topical application of TopiconDM™ insulin glargine.

FIG. 12 shows the localization at the dermal-epidermal junction of glargine microprecipitates in a representative hairless rat, formed at the site of topical application of TopiconDM™.

FIG. 13 shows a bar graph of percentage of cell viability after exposure to a TopiconDM™ patch formulation as compared to untreated control tissues.

FIG. 14 is a graph that shows rapid appearance in and elimination of SC injected glargine from the systemic circulation reciprocal blood glucose levels in hairless rats with type 1 diabetes.

FIG. 15 is a graph that shows how patch delivery can achieve insulin glargine bioavailability and induce a therapeutic response comparable to SC injection.

FIG. 16 shows results of a study of insulin glargine blood glucose lowering effects comparing delivery by patch or SC injection.

FIG. 17 shows a chart of experimentally determined hGH flux in a full thickness human skin model.

### BRIEF DESCRIPTION OF THE SEQUENCES

SEQ ID NO: 1 is an amino acid sequence of a
TRIP-Br1 decoy peptide (*Br1):
ERQIKIWFQNRRMKWKKATGCLLDDGLEGLFEDID
SEQ ID NO: 2 is an amino acid sequence of a
TRIP-Br2 decoy peptide (*Br2):
ERQIKIWFQNRRMKWKKTGFLTDLTLDDILFADID

### DEFINITIONS

This disclosure can be better understood with reference to the following definitions:

As used herein, the singular form “a”, “an” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the terms “comprises,” “comprising,” “containing,” “having,” and the like can have the meaning ascribed to them under U.S. patent law and can mean “includes,” “including,” and the like.

Unless specifically stated or obvious from context, the term “or,” as used herein, is understood to be inclusive.

The term “perforation” shall be understood to refer to a series of holes made into a material that allows easy separation of two sections of the material. The holes may be circular or may be elongated. The process of creating perforations involves puncturing the material with a tool. Perforations can be formed by a hole punch or a cutting edge that includes “nicks,” (i.e., indentations) where the two sections of the material are not separated. Alternately, perforations can be made by a cutting wheel or a grinding wheel that includes nicks in the wheel's circumference.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THIS DISCLOSURE

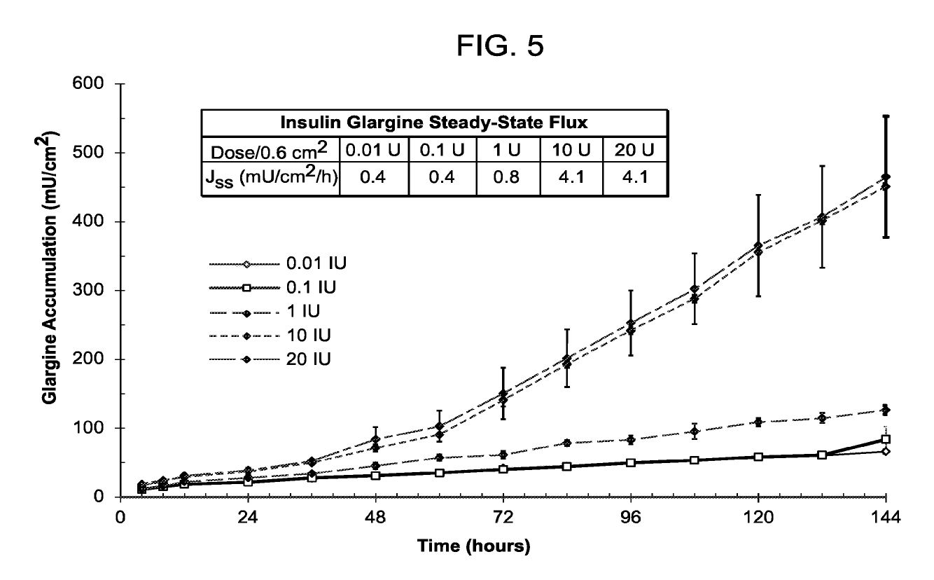

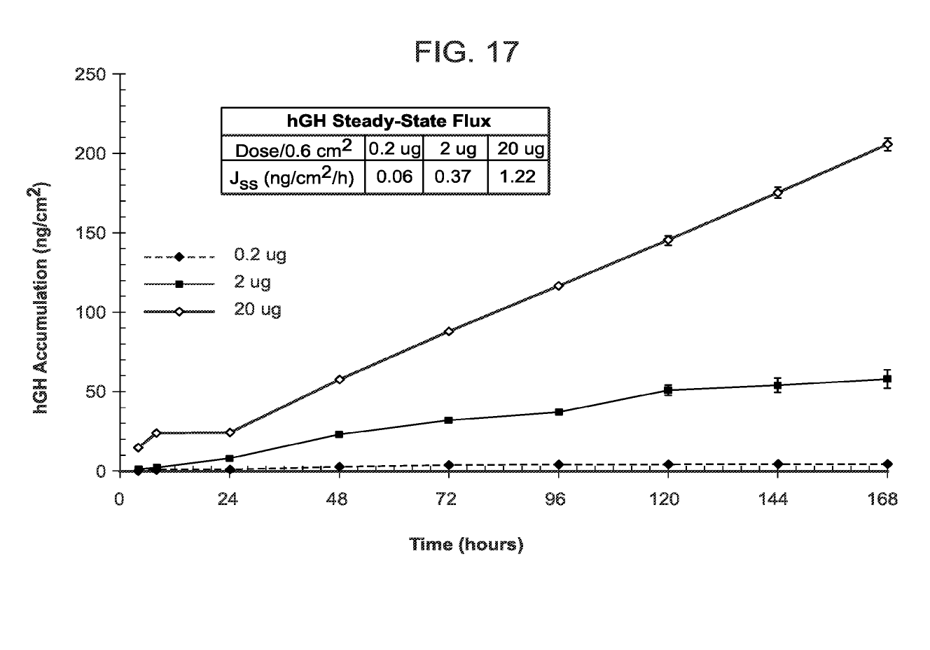

Overcoming the size barrier with novel patch formulations that allow large perishable peptides or proteins drugs such as insulin and insulin analogues (5.8-6.0 kDa; FIGS. 5-10) and human growth hormone (22 kDa; FIG. 17) to be passively delivered transdermally across full thickness (stratum corneum and viable epidermis) would dramatically increase the number of approved drug patches as a needle-free alternative to injectable drug administration. Overcoming the physicochemical constraints of transdermal patch delivery of small chemical drugs would likewise allow a multitude of such drugs to be developed. Taken together, a patch formulation that can overcome such barriers to passive transdermal drug delivery would exponentially increase the overall global drug delivery market and the transdermal drug patch market share.

The majority of current approaches under development to overcome these barriers to transdermal drug delivery use physical disruption methods that involve either mechanically removing or piercing through the stratum corneum (e.g. microneedles, thermal ablation, laser-assisted delivery, and jet injector systems) or iontophoresis-based technology. All of the technologies that are based on physical disruption are invasive, costly and bulky hardware-based technologies that may be associated with varying levels of pain, discomfort and inconvenience to patients. Iontophoresis has limited application to the delivery of small charged molecules and a maximum drug delivery rate that determined by the highest current that can be used without causing skin irritation and pain caused by the general inability of iontophoresis to localize its effects to the stratum corneum.

A variety of devices detect and continuously record a specific physiologic signal. Such devices are increasingly designed to allow for continuous ambulatory monitoring. An example is the Holter monitor, a portable device for continuous recording of various electrical activity of the cardiovascular system for at least 24 hours (often for 2-weeks at a time). The Holter device is most commonly used for monitoring heart activity (electrocardiography or ECG) to detect abnormal heart rhythm(s), but it can also be used for monitoring brain activity (electroencephalography or EEG) or arterial pressure. Its extended recording period is sometimes useful for observing occasional cardiac arrhythmias or epileptic events, which would be difficult to identify in a shorter period of time. Generally, the data is not interpreted until the end of the monitoring period. Abnormalities detected by Holter monitoring may be critical for determining disease diagnosis and to dictate appropriate clinical decision-making such as initiation of drug therapy and dose adjustment.

At least one embodiment of this disclosure provides an integrated technology that combines transdermal drug delivery with ambulatory continuous monitoring of the drug itself or a surrogate biomarker or indicator that reflects a therapeutic effect of or response to the drug. A specific embodiment of this disclosure includes but is not limited to the integration of transdermal delivery of insulin using a novel patch formulation with a continuous glucose monitoring (CGM) device. Such an integrated technology that combines two separate and independent technologies would allowing insulin administration and CGM to occur through a single direct contact (bio-interface) with the surface layer of the skin (stratum corneum) by a combined “patch-monitor.” Several examples of preferred embodiments of this disclosure are described in detail as follows.

At least one embodiment of this disclosure provides co-polymer/enhancer formulations for passive topical and transdermal or transmucosal delivery (as opposed to oral, intravenous, nasal, inhaled, etc.) of large molecule drugs currently administered only by subcutaneous or intramuscular needle injection (e.g. insulin, heparin, subunit vaccines, etc.) as well as small molecule drugs with physico-chemical properties (e.g. highly hydrophilic) that have previously precluded their administration as a passive transdermal patch embodiment (e.g. estrogen, clonidine, scopolamine, nicotine, nitroglycerin, lidocaine, fentanyl, etc.). Advantageously, in the preferred embodiments, the co-polymer/enhancer formulations disclosed herein are comprised of individual components generally regarded as safe (GRAS) and are thermo-sensitive, mucoadhesive or dermoadhesive, and enhance the penetration of therapeutics across skin or mucosal surfaces. The individual components may possess two or more of these three properties (primary, secondary and/or tertiary properties). The co-polymer/enhancer formulations can be embodied as a passive transdermal delivery patch that delivers one or more therapeutics continuously for up to 7 days or more. Also provided are therapeutic uses of the co-polymer/enhancer co-polymer/enhancer formulations for non-invasive, needle-free delivery of large peptide drugs including but not limited to insulin analogues, glucagon-like peptide-1 agonists and amylin agonists, and small nucleic acid therapeutics such as microRNAs (miRNAs) and anti-sense RNAs (siRNA, shRNA, oligonucleotides, etc.).

In a preferred embodiment, the delivery system comprises co-polymers of poloxamer 188 (P188) and propylene glycol (PG), the penetration-enhancer laurocapram (Azone) and, optionally, other classes of penetration-enhancing compounds (including short penetration-enhancing peptides), and one or more therapeutic agents. In a preferred embodiment, the delivery formulation disclosed herein can be used for transdermal delivery of large molecule perishable drugs (e.g. peptides or proteins) such as insulin, glucagon-like peptide-1 agonists and amylin agonists that are approved for the treatment of diabetes and are all administered by subcutaneous needle injection. In a preferred embodiment, the delivery formulation disclosed herein can be used to deliver regular insulin (unmodified human recombinant insulin) or rapid-acting (insulin lispro, insulin aspart, or insulin glulisine) or long-acting (insulin glargine or insulin detemir) insulin analogues.

In a preferred embodiment, the delivery system comprises co-polymers of poloxamer 188 (P188) and propylene glycol, the penetration-enhancer laurocapram (Azone) and, optionally, other classes of penetration-enhancing compounds (including short penetration-enhancing peptides), and one or more therapeutic agents. In a preferred embodiment, the delivery formulation disclosed herein can be used for topical delivery of large peptides, such as TRIP-Br decoy peptides including the TRIP-Br1 decoy peptide (*Br1) and/or the TRIP-Br2 decoy peptide (*Br2). Successful transdermal delivery of fluorescently conjugated *Br1 was achieved across a mouse cervical squamous epithelium. TRIP-Br decoy peptides target disruption of the TRIP-Br integrator function at E2F-responsive promoters, which induces a novel mechanism of cell death in proliferating cells (8-10); therefore, topical delivery of the TRIP-Br decoy peptides disclosed herein not only provides local therapy of pre-cancerous cervical dysplasia, but also prevents progression of pre-cancerous conditions to cervical cancer.

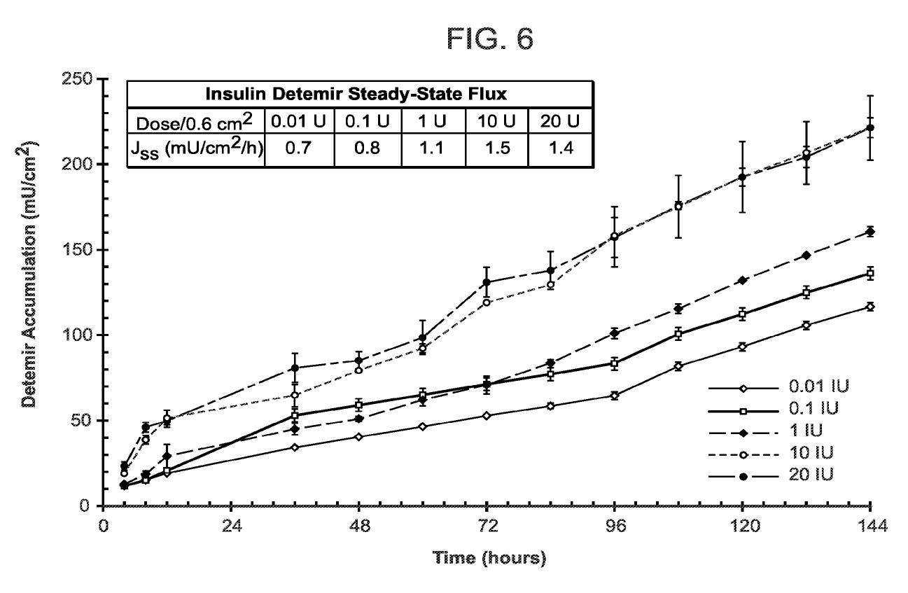

As demonstrated in FIG. 6, the co-polymer/enhancer formulations disclosed herein are thermo-sensitive; that is, solid at room temperature for ease of application to the skin (30-32° C.; 50% poloxamer 188 and 50% propylene glycol) or insertion into the cervical transformation zone (core body temperature of 37° C.; 70% poloxamer 188 and 30% propylene glycol) and transition (melt) to a gel or liquid phase at physiological temperatures. Furthermore, the co-polymer/enhancer formulations adhere to and enhance large and small molecule penetration across the skin or cervical mucosa.

Embodiments of this disclosure can be used for topical therapy of human HPV-associated low-grade cervical dysplasia defined cytologically as cervical intraepithelial neoplasia stage I or II (CIN I, CIN II) or for high-grade dysplasia (CIN III) to prevent progression to carcinoma in situ (CIS) and invasive cervical cancer (11).

Advantageously, the co-polymer/enhancer formulations disclosed herein allow for non-invasive, targeted delivery of therapeutics across the skin and mucosal surfaces. Additionally, the present co-polymer/enhancer formulations use safe, inexpensive ingredients, are easy to administer and are suitable for use in a wide range of clinical settings. The co-polymer/enhancer formulations can easily be administered by healthcare workers or by self-administration by the patient, and under conditions of extreme temperature, high humidity, poor lighting, lack of space or lack of adequate supply of electricity or water.

### Co-Polymer/Enhancer Formulation for Topical Delivery of Therapeutic Compounds

One aspect of this disclosure provides co-polymer/enhancer formulations for topical delivery of therapeutics. Advantageously, the co-polymer/enhancer formulations of this disclosure are thermo-sensitive, mucoadhesive or dermoadhesive, and enhance the penetration of therapeutics across the full thickness of the skin or mucosal surfaces.

In one embodiment, the novel topical delivery formulation comprises a thermo-sensitive polymer, a mucoadhesive or dermoadhesive polymer, a penetration enhancer and, optionally, one or more therapeutic agents.

In one embodiment, the co-polymer/enhancer formulation comprises co-polymer of one or more thermo-sensitive polymers, one or more mucoadhesive or dermoadhesive polymers and one or more penetration-enhancing agents. In a preferred embodiment, the co-polymer/enhancer formulation comprises co-polymer of poloxamer 188 and propylene glycol, the penetration-enhancer laurocapram and, optionally, one or more therapeutic agents.

In one embodiment, the co-polymer/enhancer formulation comprises one or more polymeric materials including, but not limited to, poloxamer and poloxamine. Poloxamers useful according to this disclosure include, but are not limited to, poloxamer 188, 407, 101, 105, 108, 122, 123, 124, 181, 182, 183, 184, 185, 212, 215, 217, 231, 234, 235, 237, 238, 282, 284, 288, 331, 333, 334, 335, 338, 401, 402, and 403. Poloxamines useful according to this disclosure include, but are not limited to, poloxamine 304, 504, 701, 702, 704, 707, 901, 904, 908, 1101, 1102, 1104, 1301, 1302, 1304, 1307, 1501, 1502, 1504, and 1508.

In certain embodiments, the co-polymer/enhancer formulation can comprise one or more polymeric materials including, but not limited to, polylactic acid and copolymers, polyvinyl acetate, celluloses and derivatives (such as carboxymethyl cellulose, cellulose acetate, cellulose acetate propionate, ethyl cellulose, hydroxypropyl methyl cellulose, hydroxyalkyl methyl celluloses and alkyl celluloses), crosslinked dextrans, polyethylene glycol, diethylaminoethyl dextran, poly(cyanoacrylates), copolymers of PEG and PLA, poly(lactic-co-glycolic acid), poly(ortho esters) and hydrogels. Preferably, the polymeric material is pharmaceutically-acceptable, biodegradable, mucoadhesive or dermoadhesive and/or enhances the penetration of therapeutics across the skin and/or mucosal surface.

In one embodiment, the co-polymer/enhancer formulation further comprises one or more mucoadhesive or dermoadhesive agents. In one embodiment the mucoadhesive or dermoadhesive agent promotes adhesion of the co-polymer/enhancer formulation to the skin or mucosa membranes, e.g., cervical epithelium. Preferably, the mucoadhesive or dermoadhesive agent also enhances the penetration of therapeutics across the skin and/or mucosal surface.

Mucoadhesive or dermoadhesive agents useful according to this disclosure include, but are not limited to, polyols such as, propylene glycol, dipropylene glycol, polyethylene glycol, glycerine and butylene glycol; glycol derivatives with glycerol esters, such as, oleic acid esters of propylene glycol; and non-ionizable glycol ether derivatives, such as, ethoxydiglycol.

Mucoadhesive or dermoadhesive agents useful according to this disclosure, can also include polymers such as, polyethylene glycol caprylic/capric glycerides; vinyl polymers (e.g., polyhydroxyethyl acrylate, polyhydroxyethyl methacrylate, polyvinyl alcohol and polyvinyl pyrrolidone); cellulosic derivatives, such as, methyl cellulose, ethyl cellulose, hydroxyethyl cellulose, hydroxypropyl cellulose, hydroxypropylmethyl cellulose and carboxymethyl cellulose; polysaccharides, such as, alginic acid and sodium alginate.

In one embodiment, the topical delivery formulation further comprises one or more penetration enhancers. Penetration enhancers useful according to this disclosure include, but are not limited to, laurocapram, diethylene glycol, monoethyl ether, n-decyl methyl sulfoxide, dimethyl sulfoxide, dimethylacetamidedimethylformamide, sucrose monooleate, amides and other nitrogenous compounds (e.g., urea, 2-pyrrolidone, 1-methyl-2-pyrrolidone, ethanolamine, diethanolamine and triethanolamine), organic acids (e.g., citric acid and succinic acid), N-methyl-2-pyrrolidine, borage oil, tetrahydropiperine (THP), alcohols (e.g., methanol, ethanol, propanol, octanol, benzyl alcohol, myristyl alcohol, cetyl alcohol, stearyl alcohol), fatty acids (e.g., oleic acid), fatty acid esters (e.g., isopropyl myristate, isopropyl palmitate), polyols (e.g., propylene glycol, polyethylene glycol, glycerol), polyethylene glycol monolaurate and lecithin.

In one embodiment, the penetration modifier can either enhance or retard penetration when combined with specific mucoadhesive or dermoadhesive agents such that, for example, it acts as a penetration enhancer in combination with propylene glycol but it acts as a penetration retardant in combination with polyethylene glycol. A co-polymer/retardant formulation may be used to prevent penetration across the skin or mucosal surfaces of harmful compounds including but not limited to toxins released during an environmental accident or catastrophe. In this embodiment, such co-polymer/retardant formulations may serve as a form of personal protection or as a medical countermeasure (MCM) for chemical, biological, radiological, and nuclear agents, as well as the for infectious agents, pandemic influenza and other emerging infectious diseases. In another embodiment, such co-polymer/retardant formulations may serve to prevent pesticide poisoning through exposed skin of agricultural farm workers.

Preferably, the co-polymer/enhancer formulation is solid or semi-solid at room temperature and melts at a temperature slightly below physiological temperatures. Generally, room temperature is below 30° C., below 28° C., below 25° C., below 23° C., below 20° C., or below 18° C.

In certain embodiments, the co-polymer/enhancer formulation melts, or begins to melt, at a temperature ranging from about 30° C. to 42° C., 32° C. to 40° C., 33° C. to 40° C., 35° C. to 38° C., or 34° C. to 37° C. In certain embodiments, the co-polymer/enhancer formulation melts, or begins to melt, at a temperature above 30° C., 31° C., 32° C., 33° C., 34° C., 35° C., 36° C., or 37° C. In certain embodiments, the biopolymer formulation melts, or begins to melt, at a temperature below 45° C., 44° C., 43° C., 42° C., 41° C., 40° C., 39° C., 38° C., 37° C., 36° C., 35° C., or 34° C.

The desired thermal property of the co-polymer/enhancer formulation can be achieved by adjusting the relative ratio (e.g., in terms of weight percentages or molar amounts) of various ingredients including, the thermo-sensitive polymeric material, the mucoadhesive agent, the penetration enhancer and/or the therapeutic agent.

In certain embodiments, the co-polymer/enhancer formulation comprises a polymeric material at a weight percentage of about 20% to about 95%, about 25% to about 90%, about 30% to about 85%, about 35% to about 80%, about 40% to about 70%, about 50% to about 90%, about 50% to about 85%, about 60% to about 80%, about 30% to about 40%, about 30% to about 50%, about 70% to about 90%, about 70% to about 85% or about 70% to about 80%.

In certain embodiments, the co-polymer/enhancer formulation comprises a mucoadhesive or dermoadhesive agent at a weight percentage of about 5% to about 90%, about 10% to about 80%, about 10% to about 70%, about 10% to about 60%, about 10% to about 50%, about 10% to about 40%, about 10% to about 35%, about 10% to about 30%, about 10% to about 20%, about 5% to about 30%, about 5% to about 20%, about 5% to about 15% or about 15% to about 30%.

In certain embodiments, the co-polymer/enhancer or co-polymer/retardant formulation comprises a penetration enhancer or penetration retardant, respectively, at a concentration ranging from about 0.1 M to about 1 M, about 0.2 M to about 0.9 M, about 0.3 M to about 0.8 M, about 0.4 M to about 0.7 M, or about 0.2 M to about 0.5 M.

In certain embodiments, the co-polymer/enhancer or co-polymer/retardant formulation comprises a penetration enhancer or penetration retardant, respectively, at a concentration above 0.05 M, 0.1 M, 0.15 M, 0.2 M, 0.3 M, 0.4 M, 0.5 M, 0.6 M, 0.7 M, 0.8 M, 0.9 M, 1.0 M, 1.5 M, 2 M, 2.5 M or 3 M.

In certain embodiments, the co-polymer/enhancer or co-polymer/retardant formulation comprises a penetration enhancer or penetration retardant, respectively, at a concentration below 7 M, 6 M, 5 M, 4.5 M, 4 M, 3.5 M, 3 M, 2.5 M, 2 M, 1.5 M, 1 M, 0.9 M, 0.8 M, 0.7 M, 0.6 M or 0.5 M.

In certain specific embodiments, the co-polymer/enhancer formulation comprises poloxamer 188 and propylene glycol at a ratio (w/w) of about 100:0, 90:10, 80:20, 70:30, 60:40, 50:50, 40:60, 30:70, 20:80, 10:90, or 0:100. In preferred embodiments, the co-polymer/enhancer formulation comprises poloxamer 188 and propylene glycol at a ratio (w/w) of about 70:30 (37° C. or core body temperature) or 50/50 (30-32° C. or skin temperature).

In certain embodiments, the co-polymer/enhancer formulation comprises laurocapram at a concentration of about 0.1 M to about 1 M, about 0.2 M to about 0.9 M, about 0.3 M to about 0.8 M, about 0.4 M to about 0.7 M or about 0.2 M to about 0.5 M. In a preferred embodiment, the co-polymer/enhancer formulation comprises about 0.4 M laurocapram.

The co-polymer/enhancer formulation can be used for topical delivery of a variety of small or large therapeutic agents not previously achieved using penetration enhancers including, but not limited to, large peptides and proteins greater than 53 amino acids, nucleic acids, compounds with unique physicochemical structures and/or properties not considered amenable to passive transdermal or transmucosal delivery, chemotherapeutic agents, anti-cancer or anti-tumor agents, antibiotics, anti-bacterial agents, anti-viral agents, anti-fungal agents, anti-microbial agents, anti-neoplastic agents, immunomodulatory agents, anti-inflammatory agents, cytokines and chemokines (e.g. interleukins), agents suitable for the treatment of diabetes such as insulin preparations with or with out secretagogues and/or thiazolidinediones, agents suitable for acute and/or chronic anti-coagulation (e.g. low-molecular weight heparin), vaccine antigens and hormones currently administered by subcutaneous or intramuscular needle injection or intravenous delivery through an indwelling catheter. In a preferred embodiment the co-polymer/enhancer formulations are useful for topical delivery of therapeutic agents for treatment of cervical dysplasia and for transdermal or transmucosal delivery of large therapeutic biomolecules such as insulin, anti-thrombotic agents and vaccine antigens.

Conversely, co-polymer/retardant formulations can be used to prevent penetration across the skin or mucosal surfaces of harmful compounds including but not limited to toxins released during an environmental accident or catastrophe. In this embodiment, such co-polymer/retardant formulations may serve as a form of personal protection or as a medical countermeasure (MCM) for chemical, biological, radiological, and nuclear agents, as well as the for infectious agents, pandemic influenza and other emerging infectious diseases. In this embodiment, such co-polymer/retardant formulations may also serve to prevent pesticide poisoning through exposed skin of agricultural farm workers.

Examples of decoy peptides useful in the disclosed formulations include, but are not limited to, TRIP-Br decoy peptides including TRIP-Br1 decoy peptide (*Br1) (ATGCLLDDGLEGLFEDID) (SEQ ID NO: 1) and TRIP-Br2 decoy peptide (*Br2) (TGFLTDLTLDDILFADID) (SEQ ID NO: 2), which are described in U.S. Pat. No. 6,998,383 (see claims 1-5).

Examples of chemotherapeutics and anti-cancer/anti-tumor agents useful in the disclosed formulations include, but are not limited to, 5-fluorouracil, chlorambucil, aminolevulinic acid, altretamine, ambomycin, vincristine, buthionine sulfoximine, asparaginase, bleomycin, busulin, trimetrexate, adriamycin, taxotere, carboplatin, cispiatin, carmustine, cladribine, 5-ethynyluracil, 9-dihydrotaxol, mitomycin, abiraterone, acivicin, teniposide, aclarubicin, acodazole hydrochloride, canarypox IL-2, acronine, thioguanine, acylfulvene, adecypenol, adozelesin, aldesleukin, thiotepa, ambamustine, busulfan, ametantrone acetate, amidox, amrubicin, mercaptopurine, cyclophosphamide, cytarabine, paclitaxel, pentostatin, dacarbazine, dactinomycin, daunorubicin, camptothecin derivatives, doxorubicin, etoposide, fludarabine phosphate, hydroxyurea, BRC/ABL antagonists, breflate, brequinar sodium, bropirimine, budotitane, amifostine, actinomycin, calcipotriol, calphostin C, calusterone, caracemide, carbetimer, floxuridine, idarubicin, ifosfamide, lomustine, mechlorethamine, melphalan, methotrexate, mitoxantrone, pliamycin, procarbazine, streptozocin and vinblastine.

In one embodiment, vaccine antigens can be, for example, tumor antigens or antigens from pathogenic organisms, such as viruses, bacteria, fungi and parasites. Thus, in some embodiments, the antigen is derived from a virus such as such but limited to, for example, hepatitis A virus (HAV), hepatitis B virus (HBV), hepatitis C virus (HCV), herpes simplex virus (HSV), human immunodeficiency virus (HIV), human papillomavirus (HPV), cytomegalovirus (CMV), influenza virus (e.g., influenza A virus), ebola virus, and rabies virus. In other embodiments, the antigen is derived from a bacterium such as, for example, cholera, diphtheria, tetanus, streptococcus (e.g., streptococcus A and B), Streptococcus pneumonia (e.g. over 90 serotypes, of which 88% that cause invasive disease are included in the 23-valent polysaccharide vaccine), pertussis, Neisseria meningitidis (e.g., meningitis A, B, C, W, Y), Neisseria gonorrhoeae, Helicobacter pylori, and Haemophilus influenza (e.g., Haemophilus influenza type B) and mycobacteria (e.g. Mycobacterium tuberculosis). In still other embodiments, the polypeptide-containing antigen is derived from a parasite such as, for example, a malaria parasite. Other antigens include those used to immunize against childhood diseases, such as polio, measles, mumps and rubella.

In one embodiment, hormones can include but not be limited to human or bovine growth hormone (hGH or bGH), insulin-like growth factor 1 (IGF-1), luteinizing hormone (LH), follicle stimulating hormone (FSH), prolactin, human chorionic gonadotropin (hCG), menotropin (also called human menopausal gonadotropin or hMG), estrogen, and testosterone.

Yet other embodiments provide for the inclusion of agents suitable for the treatment of diabetes mellitus (types 1 and 2) and those suitable for the prevention and treatment of thrombotic and pro-thrombotic conditions (e.g. deep-vein thrombosis, pulmonary embolism, chronic atrial fibrillation, prosthetic heart valves, sickle cell). Non-limiting examples of such agents for diabetes include insulin and insulin analogs, (e.g., lispro (Humalog), Humulin (Isophane and Regular), Novolog (Aspart), Levemir (Detemir), Lantus (glargine) as well as other small molecules, such as metformin, rosiglitazone, pioglitazone and combinations containing such molecules (e.g, metformin and rosiglitazone, rosiglitazone and glimepiride). Other compounds that can be incorporated into the disclosed composition include: biguanides, such as metformin (Glucophage); thiazolidinediones (TZDs), such as rosiglitazone (Avandia) and pioglitazone (Actos); sulfonylureas, such as tolbutamide, (Orinase), acetohexamide (Dymelor), tolazamide (Tolinase), chlorpropamide (Diabinese), glipizide (Glucotrol), glyburide (Diabeta, Micronase, Glynase), glimepiride (Amaryl) or gliclazide (Diamicron); Nonsulfonylurea secretagogues, such as meglitinides (e.g., repaglinide (Prandin) and nateglinide (Starlix); and alpha-glucosidase inhibitors, such as miglitol (Glyset) or acarabose (Precose, Glucobay); glucogon-like peptide 1 (GLP-1) agonists such as exenatide (Byetta, Bydureon), liraglutide (Victoza), and albiglutide; dipeptidyl peptidase-4 (DPP-4) inhibitors such as sitagliptin (Januvia, Javumet, Janumet XR, Javisync), vildagliptin (Galvus), linagliptin (Tradjenta, Jentadueto) saxagliptin (Onglyza, Kombiglyze XR), and alogliptin (Nesina, Kazano, Oseni); sodium glucose co-transporter 2 (SGLT2) inhibitors such as canagliflozin (Invokana), dapagliflozin (Farxiga), empagliflozin (BI10773), ipragliflozin and luseogliflozin. Non-limiting examples of such agents for prevention or treatment of acute and/or chronic pre-thrombotic or thrombotic conditions include unfractionated and low molecular weight heparins such as ardeparin (Normiflo), bemiparin (Hibor, Ivor, Zibor, Badyket), certoparin (Sandoparin), dalteparin (Fragmin), enoxaparin (Lovenox, Clexane), nadroparin (Fraxiparin), parnaparin (Fluxum), reviparin (Clivarin) and tinzaparin (Innohep, Logiparin); Factor Xa inhibitors such as fondaparinux (Arixtra) and idraparinux sodium (SANORG 34006, SR 34006), rivaroxaban (BAY 59-7939, Xarelto), and apixaban (Eliquis); direct thrombin inhibitors such as lepirudin (Refludan), bivalirudin (Angiomax or Angiox), argatroban (Acova, Arganova, Argatra, Novastan) and dabigatran (Pradaxa, Pradax, Prazaxa); and vitamin K antagonists such as warfarin (Coumadin), Acenocoumarol (Sintrom, Sinthrome) and Phenprocoumon (Marcoumar, Marcumar, Falithrom). In certain embodiments, the co-polymer/enhancer formulation comprises a therapeutic agent at a concentration ranging from about 0.1 mM to about 3 mM, about 0.1 mM to about 2 mM, about 1 mM to about 1.5 mM, about 0.5 mM to about 2 mM, or about 0.5 mM to about 1.5 mM. Amounts of therapeutic agents incorporated into co-polymer/enhancer formulations disclosed herein can also be determined by those skilled in the art (e.g., based upon age, bioavailability of a therapeutic agent, etc.) such that the therapeutic agent is delivered to a subject in amounts that effect a therapeutic benefit to the subject.

### Topical Delivery of Broad Range of Compounds

Another aspect of this disclosure provides methods for enhanced delivery of compounds (e.g., therapeutic agents) across the skin and/or mucosal surface. Embodiments of this disclosure enhances topical, non-invasive delivery of a broad range of small and large therapeutic agents of various classes and compositions as well as physicochemical properties (e.g. peptides, proteins, chemicals, nucleic acids), such as but not limited to cytotoxic decoy peptides, other chemotherapeutics and anti-tumor/anti-cancer agents, drugs for the treatment of diabetes mellitus, prevention and treatment of thrombotic and pro-thrombotic conditions, antigens for the induction of a protective immune response to various vaccine antigens, and hormones as disclosed above.

In one embodiment the method comprises administering, to skin or mucosal surface of a subject, a co-polymer/enhancer formulation of this disclosure using any standard topical patch design or variations thereof, having in common the direct application (bio-interface) with the skin or mucosal surface. In a specific embodiment the method comprises administering, to skin or mucosal surface of a subject, a co-polymer/enhancer formulation comprising poloxamer 188 and propylene glycol, laurocapram and, optionally, one or more therapeutic agents.

The term “subject,” as used herein, describes an organism, including mammals such as primates, to which treatment with the formulations according to the subject disclosure can be provided. Mammalian species that can benefit from the disclosed methods of treatment include, but are not limited to, apes, chimpanzees, orangutans, humans, monkeys; and domesticated animals such as dogs, cats, horses, cattle, pigs, sheep, goats, chickens, mice, rats, guinea pigs, and hamsters.

In certain embodiments the co-polymer/enhancer formulation of this disclosure is administered to skin or mucosal surfaces including, but not limited to, cervix, vagina, anus, rectum, eye, ear, nose, thorax, vulva, larynx, and head and neck. In one embodiment the co-polymer/enhancer formulation of this disclosure is topically administered to cervical dysplastic lesions of the ectocervix and proximal endocervical canal of women via an intravaginal route of mucosal delivery.

Embodiments of this disclosure allow for topical delivery of therapeutics across keratinized apical layer of skin (stratum corneum) and/or mucosa. At least one embodiment allows for topical delivery of therapeutics across non-keratinized surface of skin and/or mucosa. At least one embodiment allows for topical delivery of therapeutics into, or across, multiple layers of cervical squamous epithelial cells. At least one embodiment allows for topical delivery of therapeutics to the basal keratinocytes of skin and/or mucosa.

At least one embodiment can be used to deliver therapeutics to penetrate the keratinized surface of the mouse cervical transformation zone. At least one embodiment can be used to deliver therapeutics to penetrate non-keratinized human cervical transformation zone (T-zone) across multiple layers of squamous epithelial cells to reach the basal keratinocytes where HPV viral integration occurs.

As exemplified in Example 1, the co-polymer/enhancer formulations of this disclosure can deliver moderate sized peptides across the keratinized apical layer of mouse cervical T-zone. Mouse cervical transformation zone differs from that of human. Mouse cervix is completely internalized, whereas human has an ectocervix located on the vaginal surface. In addition, mouse cervix has a keratizined apical layer in the cervical T-zone, which begins from the vagino-cervical junction and ends at the squamo-columnar junction. Cervical T-zone is where most cervical cancers arise. The mouse keratinized cervical epithelium is comparable to keratinized skin. As exemplified in Example 1, the co-polymer/enhancer formulation is capable of topical delivery and penetration of a 35 amino acid cytotoxic peptide (*Br1) across the mouse keratinized cervical squamous epithelium.

At least one embodiment can be used for topical delivery of therapeutics across the keratin-rich, stratum corneum of skin to treat cutaneous neoplastic and non-neoplastic proliferative diseases including, but not limited to, HPV-associated pre-cancerous and cancerous conditions affecting vulva, vagina, anus, larynx, and head and neck, and melanoma, basal cell carcinoma, nasophyngeal carcinoma associated with Epstein-Barr Virus infection and psoriasis.

At least one embodiment can be used for topical delivery of chemotherapeutic and anti-cancer/anti-tumor agents for treatment of tumor or cancer including, but not limited to, human HPV-associated cervical cancer and its precursor lesions, such as pre-cancerous low-grade cervical dysplasia classified as cervical intraepithelial neoplasia stage I or II (CIN I, CIN II), high-grade dysplasia (CIN III), carcinoma in situ (CIS) and locally invasive or metastatic cervical cancer (11); prostate cancer; ovarian cancer; vulvar cancer; vaginal cancer or tumor; endometrial cancer; laryngeal carcinoma; nasal pharyngeal carcinoma; bladder cancer; nasopharyngeal carcinoma, skin cancer; and head and neck cancer.

At least one embodiment can be used for topical, non-invasive delivery of therapeutics for treatment of diseases or conditions, including hyperplastic skin lesions, such as, genital warts, psoriasis and keloids.

At least one embodiment can be used as a non-invasive topical transdermal or transmucosal delivery system (or device) applied to normal skin or mucosal surfaces to obviate the need for subcutaneous injection of therapeutic compounds.

### Formulations and Formulations for Topical Administration

The subject disclosure also provides for therapeutic or pharmaceutical formulations comprising the co-polymer/enhancer formulation in a form that can be combined with a pharmaceutically acceptable carrier. In a preferred embodiment the therapeutic or pharmaceutical formulation is solid at room temperature and transitions to a gel or liquid at desired physiological temperatures.

The term “carrier” refers to a diluent, adjuvant, excipient or vehicle with which the compound is administered. Such pharmaceutical carriers can be sterile liquids, such as water and oils, including those of petroleum oil such as mineral oil, vegetable oil such as peanut oil, soybean oil and sesame oil, animal oil or oil of synthetic origin.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The therapeutic formulation, if desired, can also contain minor amounts of wetting, emulsifying or pH buffering agents. These formulations can take the form of creams, foam, patches, lotions, drops, sprays, gel, oils, aerosol, powders, ointment, solutions, suspensions, emulsion and the like. The formulation can be formulated with traditional binders and carriers such as triglycerides. Examples of suitable pharmaceutical carriers are described in “Remington's Pharmaceutical Sciences” by E. W. Martin. Such formulations contain a therapeutically effective amount of the therapeutic formulation, together with a suitable amount of carrier so as to provide the form for proper administration to the patient. The formulation should suit the mode of administration.

The subject disclosure also provides for the modification of the ingredient such that it is more stable once administered to a subject, i.e., once administered it has a longer time period of effectiveness as compared to the unmodified form. Such modifications are well known to those of skill in the art, e.g., microencapsulation, etc.

The amount of the therapeutic or pharmaceutical formulation of this disclosure which is effective in the treatment of a particular disease, condition or disorder will depend on the route of administration and the seriousness of the disease, condition or disorder and should be decided according to the judgment of the practitioner and each patient's circumstances.

Further, at least one embodiment provides kits containing therapeutic agents such as, lyophilized cytotoxic decoy peptides, vehicle and/or co-polymer/enhancer formulations. Preferably, the formulations of this disclosure are stable in a wide range of temperatures below the desired melting temperature. In one embodiment the active therapeutic agents can be reconstituted by mixing pre-measured quantities of each component immediately prior to use.

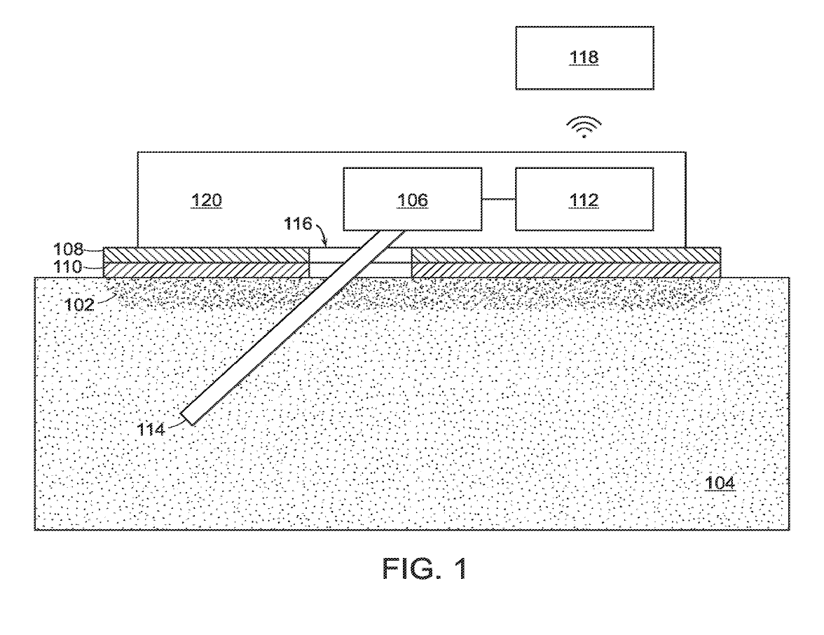

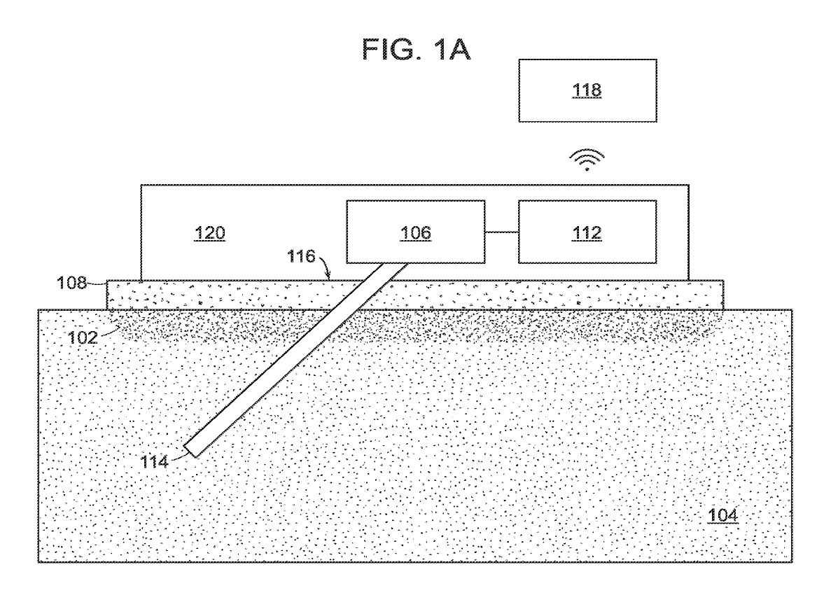

Referring now to FIGS. 1 and 1A, a device 100 allows for measurement and monitoring of a subject simultaneously with transdermal delivery of a therapeutic agent at a contact site 102 with the subject's skin 104. The device 100 includes a sensor 106, a drug formulation storage reservoir 108, a dermoadhesive agent 110, and a transmitter 112. In some embodiments, the drug storage reservoir 108 is a thermo-sensitive co-polymer/enhancer matrix in a solid state at ambient temperature (e.g. room temperature, 25° C.), which transitions to a dermoadhesive gel phase when the device is applied to the bio-interface site and reaches skin temperature (30-32° C.). In such an embodiment, as shown in FIG. 1A, the drug storage reservoir 108 can include the dermoadhesive as part of the thermo-sensitive co-polymer/enhancer matrix described above, which serves as the dermoadhesive upon transition to gel phase.

The sensor 106 is adapted and configured to detect a specific indicator that is either the therapeutic agent itself or a biomarker that is affected by (e.g., directly, indirectly, or otherwise serves as an indicator of agent function) the therapeutic agent. The sensor 106 can be a transdermal sensor that detects a specific indicator with or without piercing the subject's skin. In addition to the invasive interstitial sensors described below, transdermal sensors can utilize non-invasive technology such as near-infrared (NIR) and sweat analysis.

The sensor 106 can include one or more electrodes 114. The electrodes 114 can be adapted and configured to penetrate across the subject's full thickness skin 104, for example, to obtain measurements from the subepidermal interstitial space. For example, a leading end of the electrode(s) 114 can be beveled to facilitate piercing of the skin 104. Additionally or alternatively, the electrode(s) 114 can be bent or curved to facilitate extension into the skin, while allowing the bio-interface surface of the device 100 to make full contact flush against the subject's skin and held in place by a dermoadhesive 110. In the preferred embodiment, the drug storage reservoir 108 and the dermoadhesive 110 comprise the thermo-sensitive co-polymer/enhancer matrix described above, which serves as the dermoadhesive 110 upon transition from solid to gel phase.

Electrodes 114 can have a variety of features to facilitate detection of various specific indicators using various chemistries. For example, a variety of chemistries for measuring glucose are described Geoffrey McGarraugh, “The Chemistry of Commercial Continuous Glucose Monitors,” 11 (Supplement 1) Diabetes Technology & Therapeutics S-17-24 (2009). In one embodiment described in U.S. Patent Application Publication No. 2012/0172691 and commercialized under the DEXCOM® SEVEN PLUS™ trademark by Dexcom, Inc. of San Diego, Calif., the electrode 114 is coupled with immobilized glucose oxidase enzyme. The immobilized glucose oxidase catalyzes the oxidation of glucose to hydrogen peroxide and D-glucono-δ-lactone. The resulting hydrogen peroxide reacts with a surface of the electrode to produce electric current that can be measured and correlated with a blood glucose level (e.g., based on a prior measurement of the subject's blood glucose level during periodic finger pricks).

In addition to the DEXCOM® SEVEN PLUS™ system and its next generation version G4 Platinum™ offered by Dexcom, Inc., other suitable continuous glucose monitoring (CGM) systems include the MINIMED PARADIGM REAL-TIME REVEL and GUARDIAN REAL-TIME CGM systems offered by Medtronic, Inc. of Minneapolis, Minn. All such continuous glucose monitors are stand-alone devices for continuous measurement of a biomarker (i.e. glucose) in patients receiving drug therapy that may include oral and/or injectable drugs such as metformin and insulin analogues, respectively, which function to achieve varying degrees of glycemic control (i.e. conventional or tight control) in diabetic subjects.

The sensor 106 or one or more portions thereof such as attached electrode(s) 114 can be disposable and biodegradable, while the transmitter 112 and the receiver can be reused.

Storage reservoir 108 and dermoadhesive 110 can be a thermo-sensitive co-polymer/enhancer matrix as described above, which serves as the dermoadhesive 110 upon transition from solid to gel phase after application to the skin surface. In this embodiment, the thermo-sensitive matrix patch can be a therapeutic-agent-containing formulation for passive or active drug delivery. The thermo-sensitive matrix patch can deliver the therapeutic agent via passive or active diffusion across a skin surface that is not being physically disrupted with technologies such as thermal or laser ablation, microneedles, high pressure jet injection, ultrasound, sonar, iontophoresis, and the like. Suitable formulations include the co-polymer/enhancer formulations described above and in International Publication No. WO 2012/135422. Such formulations can be used to deliver small molecule drugs, large molecule drugs, therapeutic peptides, DNA, and/or microRNA. Specific examples of therapeutic agents include human native insulin, insulin analogues, insulin glargine (offered under the LANTUS® trademark by Sanofi-Aventis of Paris, France), or insulin glulisine (offered under the APIDRA® trademark by Sanofi-Aventis of Paris, France). Other examples of therapeutic agents include anti-coagulants (e.g., enoxaparin offered under the LOVENOX® trademark by Sanofi-Aventis of Paris, France), chemotherapeutics (e.g. taxol-based chemotherapeutics such as cabazitaxel) offered under the JEVTANA® trademark and leuprolide offered under the ELIGARD® trademark by Sanofi-Aventis of Paris, France), anti-nausea therapeutics (e.g., dolasetron offered under the ANZEMET® trademark by Sanofi-Aventis of Paris, France), immuno stimulants (e.g., granulocyte macrophage colony-stimulating factor sargramostim offered under the LEUKINE® trademark by Sanofi-Aventis of Paris, France), vaccines (e.g., influenza vaccines offered under the FLUZONE® trademark by Sanofi-Aventis of Paris, France; tuberculosis vaccines offered under the TUBERSOL® trademark by Sanofi-Aventis of Paris, France; and human papillomavirus vaccines), and the like.

Storage reservoir 108 can include one or more openings 116 to allow passage of a sensor electrode 114. Alternatively, the storage reservoir 108 and/or the electrode(s) 114 of the sensor 106 can be configured to allow the electrode(s) 114 to easily pierce the storage reservoir 108 and the contact site 102 of the skin 104 without diminishing the performance of the electrodes 114.

Dermoadhesive agent 110 can be a dermoadhesive polymer as described above and in International Publication No. WO 2012/135422. Examples of suitable dermoadhesive agents include propylene glycol, dipropylene glycol, polyethylene glycol, glycerine, butylene glycol, glycol derivatives with glycerol esters, non-ionizable glycol ether derivatives, and the like. In some embodiments, the dermoadhesive agent is applied to storage reservoir 108 during manufacture and can be revealed and thermally activated by removal of a liner before application to the contact site 102.

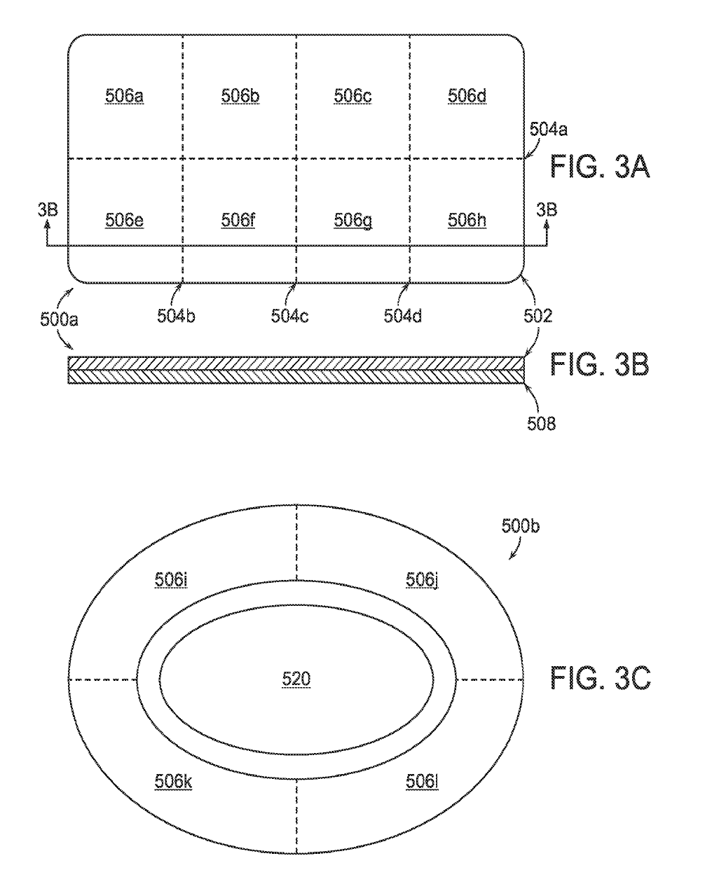

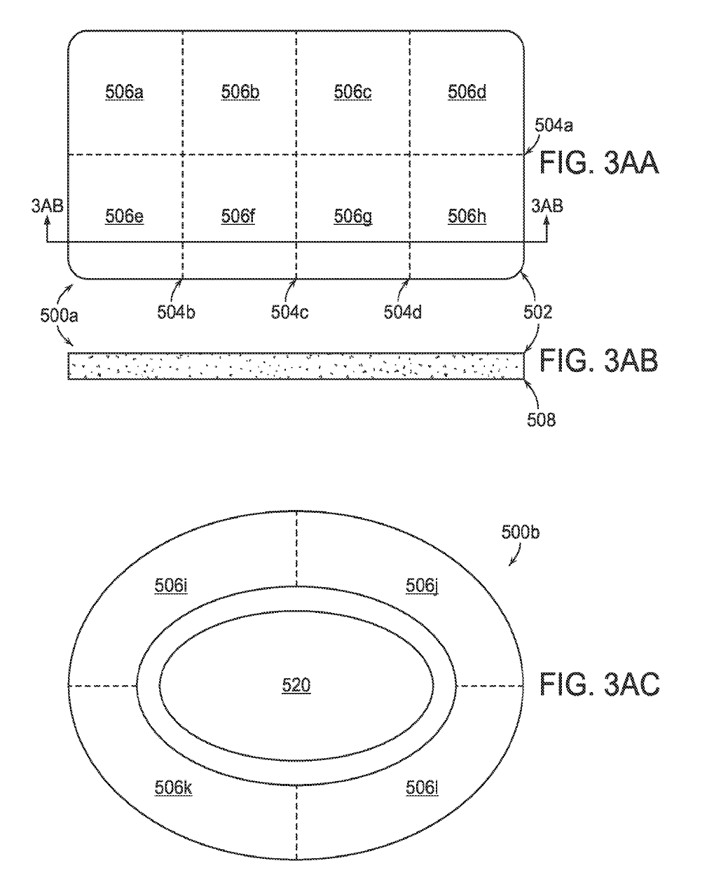

Although storage reservoir 108 and dermoadhesive agent 110 can be distinct layers in as shown in FIGS. 1 and 3B, dermoadhesive agent 110 can be integrated within a co-polymer/enhancer formulation as described herein. For example, storage reservoir 108 can include a co-polymer/enhancer formulation that includes a thermo-sensitive polymer, a dermoadhesive polymer, a penetration enhancer, and/or a therapeutic agent that constitutes a single solid phase matrix patch that serves as the dermoadhesive 110 upon transition from solid to gel phase upon application to the contact site 102.

Transmitter 112 can be communicatively coupled with the sensor 106 and adapted and configured to transmit data generated by the sensor 106 to a receiver 118. Transmitter 112 can be a wired or wireless transmitter. In some embodiments, the transmitter is adapted and configured to transmit data at a frequency between about 402 MHz and about 433 MHz as described in U.S. Patent Application Publication No. 2012/0172691, which advantageously minimizes power consumption. Alternatively, the transmitter 312 can utilize one or more wireless communication standards such as BLUETOOTH®, IEEE 802.11, IEEE 802.15.4, and the like. The BLUETOOTH® standard is discussed in Andrew S. Tanenbaum, Computer Networks 21, 310-17 (4th ed. 2003). The IEEE 802.11 standard is discussed in Andrew S. Tanenbaum, Computer Networks 292-302 (4th ed. 2003). The IEEE 802.15.4 standard is described in Yu-Kai Huang & Ai-Chan Pang, “A Comprehensive Study of Low-Power Operation in IEEE 802.15.4” in MSWiM'07 405-08 (2007). Advantageously, the use of standards such as BLUETOOTH® allow the receiver 118 to be a smartphone (e.g., an IPHONE® brand smartphone available from Apple Inc. of Cupertino, Calif.; an ANDROID™ brand smartphone available from manufacturers such as HTC Corporation of Taoyuan City, Taiwan; or a WINDOWS® brand smartphone available from Microsoft Corporation of Redmond, Wash.), or other electronic device that a user may already own and carry on a regular basis.

The receiver 118 can include hardware and/or software adapted and configured to allow for storage, display, analysis, and or subsequent transmission of the data received from the transmitter 112. For example, the transmitter 112 can transmit “raw” data regarding the electricity generated by oxidation of glucose at the electrode 114 and the receiver 118 can correlate this data to determine the subject's blood glucose level. The receiver 118 can also display a most recent blood glucose level and/or past blood glucose levels (e.g., textually and/or in a graph). The receiver 118 can also periodically forward the blood glucose levels to another party or device. For example, the receiver 118 can transmit a set of data to the subject or another party of the subject's choosing such as health care provider, physician, heath care data repository, and the like, via the Internet.

Analysis of the data received by the receiver 318 from the transmitter 312 can be performed in accordance with one or more proprietary algorithms defined by the manufacturer of the sensor 306. In some embodiments, either raw data or processed data is further analyzed (post-processed) using the Ambulatory Glucose Profile algorithm (CapTurAGP™) described in Roger Mazze et al., “An Overview of Continuous Glucose Monitoring and the Ambulatory Glucose Profile,” 94(8) Minn. Med. 40-44 (August 2011) and available for licensing for research purposes from the Park Nicollet International Diabetes Center of St. Louis Park, Minn.

The sensor 106 and the transmitter 112 can be combined within a single housing 120 that can be fabricated from various materials such as plastics, rubbers, metals, or the like. The reusable transmitter 112 can be a separate component that snaps into place on top of the disposable housing 120, as in the case of the DEXCOM® CGM devices. The housing 120 can be held against the contact site 102 by a variety of means, including chemical and/or mechanical attachment to the storage reservoir 108. The housing can also be further held against the contact site 102 by the dermoadhesive properties of the co-polymer/enhancer formulations described herein upon transition from solid to gel phase.

In some embodiments, the device 100 can utilize an architecture similar to that described in U.S. Patent Application Publication No. 2012/0172691 and commercialized under the DEXCOM® SEVEN PLUS™ trademark by Dexcom, Inc. of San Diego, Calif. in which a sensor is comprised of a carriage that carries the electrode. The sensor 106 and electrode 114 are applied to the skin contact site with a plunger/injector device that rapidly inserts the electrode into the interstitial space under the skin surface. The sensor carriage contact surface with the skin is coated with a strong adhesive 110 that is applied to the skin simultaneously with electrode insertion. The transmitter 112 is then snapped into place on top of the housing on the top of the carriage so that the transmitter 112 forms a unit with the sensor housing 120. Attachment of the transmitter 112 brings its circuitry into direct contact with that of the sensor 106. The electrode 114, sensor 106 and transmitter 112 thereby relay the interstitial glucose measurement data generated by the sensor to the receiver 118.

Storage reservoir 108 and housing 120 can preferably be maintained in place at the contact site 102 for an extended period of time during a variety of activities (e.g., exercise and sleeping). For example, the storage reservoir 108 can contain a sufficient quantity of an extended release formulation and the housing 120 can have a power source sufficient to allow both the storage reservoir 108 and the housing 120 to remain in place and fully functional for about 1 day, 2 days, 3 days, 4 days, 5 days, 6 days, 7 days, 8 days, 9 days, 10 days, 11 days, 12 days, 13 days, 14 days, 2 weeks, 3 weeks, or the like. The lifespan can differ between the storage reservoir 108 and the housing 120. In the case of the DEXCOM® CGM devices, the sensor is approved for continuous use for up to 7 days.

The sensor electrode(s) 114, sensor 106 and the transmitter 112 can collect and/or transmit data continuously, substantially continuously, or periodically. For example, data can be collected and/or transmitted at an interval T, wherein T is selected from the group consisting of: less than about 1 second, between about 1 second and about 5 seconds, between about 5 seconds and about 10 seconds, between about 10 seconds and about 30 seconds, between about 30 seconds and about 60 seconds, between about 1 minute and about 5 minutes, between about 5 minutes and about 10 minutes, between about 10 minutes and about 15 minutes, between about 15 minutes and about 30 minutes, between about 30 minutes and about 60 minutes, between about 1 and 2 hours, between about 2 and 4 hours, or the like. In the case of the DEXCOM® CGM devices, one interstitial glucose measurement is transmitted every five minutes to the receiver continuously for up to seven days.

The size of the device 100 can be varied to meet the needs of various patients. For example, a surface of the device 300 adjacent to the contact site 102 can be between about 4 and about 25 square centimeters. The volume of the device 100 can between about 4 and about 25 cubic centimeters in size. In one embodiment, the size of the storage reservoir 308 varies depending on the amount of the therapeutic agent to be administered to the subject.

Although a particular transdermal glucose sensor is described above, the combination of a transdermal sensor and a therapeutic agent delivery patch can be applied to any transdermal sensor, particularly transdermal glucose sensors useful for the monitoring and treatment of diabetes mellitus.

### Methods of Use of Combined Delivery and Monitoring Device

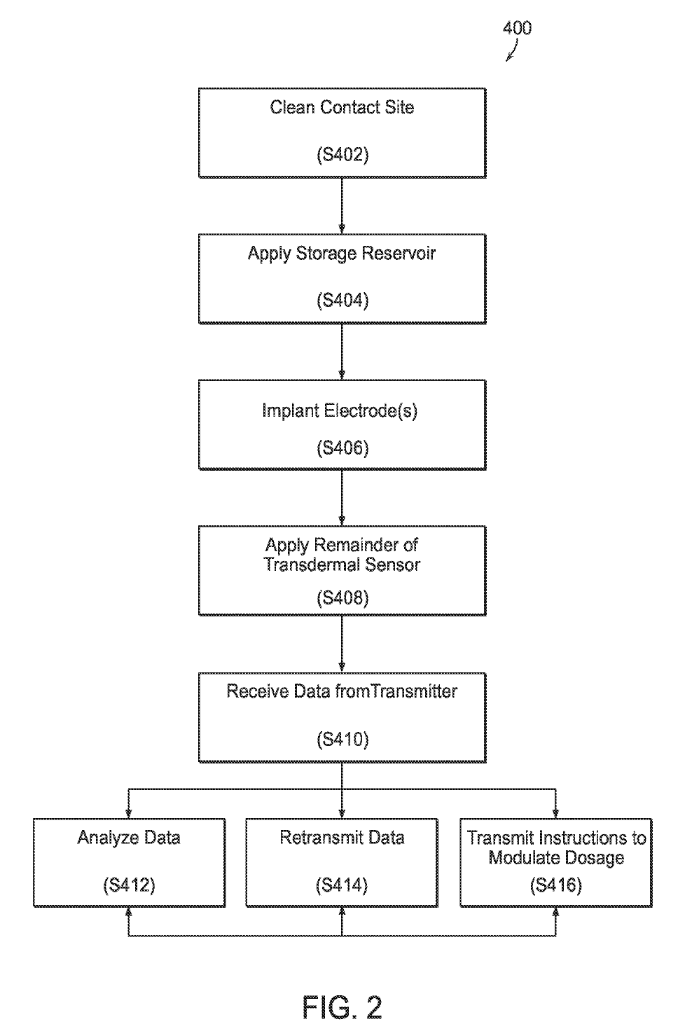

Referring now to FIG. 2, a method 400 of utilizing a combined delivery and monitoring device is described. As discussed above, the devices 100 described herein can have a variety of features and configurations. Accordingly, it will be appreciated that the method 400 can be performed without all steps.

In step S402, a contact site is cleaned (e.g., with rubbing alcohol).

In step S404, a sensor housing containing a sensor electrode(s), sensor and storage reservoir and adhesive is applied to the contact site.

In step S406, one or more electrodes are implanted. If the detection method involves insertion of an electrode(s), it can be implanted at the time of application of the storage reservoir in step S404. The entire sensor unit can be held in place by adhesive or by a belt, bandage, or similar means. In some embodiments, a plunger (e.g., as described in U.S. Patent Application Publication No. 2012/0172691 and commercialized under the DEXCOM® SEVEN PLUS™ trademark by Dexcom, Inc. of San Diego, Calif.) can be utilized to implant the electrode(s).

In step S408, any remaining portions of the transdermal sensor are applied, including the attachment of the transmitter to the sensor housing. As discussed above, any remaining portions of the transdermal sensor can be coupled to the storage reservoir.

In step S410, data is received from the transmitter. As discussed above, data can be received in a wired or wireless manner using a special purpose or a general purpose receiver such as a smartphone.

In step S412, the data is analyzed. For example, the data can be analyzed and displayed using the CapTurAGP™ to determine a subject's continuous ambulatory blood glucose profile over the past 24 hours.

In step S414, the data is retransmitted, for example, to a computing device (e.g. computer, smartphone, etc.) for long-term storage or to a physician for review and clinical decision-making regarding dose adjustments of anti-diabetic medications including oral agent or short- and long-acting insulin analogues.

In step S416, instructions to modulate a dosage of the therapeutic agent are transmitted to the subject. For example, the user can be instructed to reduce a dosage of insulin and/or drink orange juice if the received data indicates that the subject is hypoglycemic. Such instructions can be transmitted in a variety of ways. For example, the instructions can be displayed textually on the receiver and/or communicated audibly, particularly if the receiver is a smartphone or other device including a display and/or a speaker. The data loop comprised of steps S410-S416 can be performed in real-time to allow patient and physician to optimally manage glycemic control in a model referred to as a “closed loop” or “artificial pancreas,” which is considered the most desirable method of diabetes management. In such a model, CGM provides the data displayed in a manner that allows clinical decision-making regarding therapeutic dose adjustments with the goal of closely mimicking the physiologic role of the pancreas in regulating blood glucose levels through appropriate release of insulin and other counter-regulatory hormones.

### Modulatable Transdermal Patches

Referring now to FIGS. 3A-3C and FIGS. 3AA-3AC, another aspect of this disclosure provides a modulatable transdermal patch 500a that can allow a subject to modulate the dosing of a therapeutic agent (i.e make dose adjustments), for example, based on a measurement obtained from a device such as a continuous glucose monitor.

Modulatable transdermal patch 500a includes a perforated backing 502. Suitable backing materials are available but not limited to those under the COTRAN™ AND SCOTCHPAK™ trademarks from 3M Company of Maplewood, Minn. Perforations 504 (e.g., a nick or a skip score pattern) can be applied to backing 502 using various commercially available perforating machines in order to divide the patch 500a into a plurality segments 506.

A therapeutic-agent-containing formulation 508 can be applied to backing 502. The therapeutic-agent-containing formulation 508 can be a co-polymer/enhancer formulation as disclosed herein.

Referring to FIGS. 3AA-3AC, can be a single layer formed using therapeutic-agent-containing formulation 508.

Modulatable transdermal patch 500a allows a subject to modulate the dosage of a therapeutic agent by removing and/or reapplying one or more segments 506. For example, if the subject determines that their blood glucose level is too low, the subject can remove one or more segments 506 from their skin by tearing the one or more segments 506 along perforations 504. Optionally, the subject can also wipe and/or wash the contact site between the removed segment(s) and the subject's skin to remove remaining therapeutic agent from the contact site.

Referring now to FIG. 3B, another embodiment of a modulatable transdermal patch 500b is depicted. In at least one embodiment, patch 500b has an elliptical shape with a plurality of removable segments along the exterior of patch 500b. When used in connection with a sensor 520, segments 506 can advantageously be removed without disturbing or removing sensor 520 applied to the center of patch 500b.

Incorporation of Existing Pharmaceuticals into Transdermal Patches

Despite the vast number of commercially-available pharmaceuticals, very few (currently about 40) pharmaceuticals are commercially available in transdermal patch formulations. However, the transdermal patches and co-polymer/enhancer formulations described herein enable the administration of broad classes of pharmaceuticals transdermally. Examples of particular pharmaceuticals suitable to be administered transdermally using the technology described herein is provided in the Appendix.

### Application of Transdermal Patches to Diabetes Mellitus

Advantageously, the transdermal patch systems described herein can be engineered to have thermo-sensitive properties, a solution to the problem of “cold chain” storage and distribution for all liquid perishable injectable drugs (that is, difficulties in maintaining uninterrupted storage and distribution activities within specific or tightly controlled temperature ranges, humidity ranges, etc. to ensure product integrity). Embodiments of the patches described herein can be thermally profiled to maintain the stability of perishable large molecule drugs in a solid matrix at 25° C. room temperature (thereby eliminating the need for cold storage) and to melt at skin temperature (30-32° C.) into a dermoadhesive gel that enhances transdermal drug delivery.

Additionally, the transdermal patch systems described herein avoid the complications of insulin injections, including the risk of injecting insulin directly into a vein, which can cause hypoglycemia.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

Following are examples that illustrate procedures for practicing this disclosure. These examples should not be construed as limiting. All percentages are by weight and all solvent mixture proportions are by volume unless otherwise noted.

### Example 1

### Thermo-Sensitive, Mucoadhesive, and Penetration-Enhancing Formulations

To create co-polymer/enhancer formulations with desired thermo-sensitive properties, USP grade poloxamer 188 (P188) and propylene glycol (PG) were mixed at various ratios (100/0, 80/20, 70/30, 50/50 and 0/100) and heated to 60° C. The co-polymer/enhancer preparations were suctioned into pre-warmed (60° C.) Silastic tubing (⅛^{th}-inch inner diameter; Fisher, Pittsburgh, Pa.), solidified at room temperature and extruded from the tube with compressed air. 3 mm samples were cut from the solid “rope” with a scalpel.

The thermal profile of the P188/PG co-polymer preparations was determined using a DSC 6200/Exstar 6000 (Seiko Instruments) Differential Scanning calorimeter. To obtain co-polymer preparations that have the desired melting point (30-37° C.) in the presence of 1 mM of various decoy peptides, DSC 6200 was programmed with 3 cycles of heating at 10° C./min and cooling at 50° C./min, with 2 min equilibration times between heating/cooling cycles. Measurements were taken at a temperature range of −25-65° C.

The DSC curves did not exhibit dual peaks, indicating that the P188/PG co-polymer preparations were in a homogeneous phase. The co-polymer with an 80/20 ratio of P188/PG exhibited the desired thermal property, that is, solid at room temperature and instantly melted at a temperature slightly below the physiological temperature of 37° C.

The P188/PG (80/20 ratio) co-polymer was then used to deliver FITC-tagged 38 amino acid cytotoxic decoy peptides. The P188/PG co-polymer (80/20 ratio) alone was unable to penetrate the keratinized apical layer of the mouse cervix, as determined by direct visualization using fluorescence microscopy (data not shown).

Laurocapram is a non-toxic enhancer of cutaneous penetration (13, 14). It is among a class of “penetration modifiers” that, depending on the vehicle formulation, can enhance or retard the penetration of human stratum corneum. DSC and spectral analysis revealed that penetration modifier formulations (e.g., laurocapram) disrupt and fluidize the stratum corneum lipid bilayers. Since 1984, laurocapram has been widely used as a safe penetration enhancer and has been formulated with propylene glycol—a mucoadhesive and dermoadhesive agent.

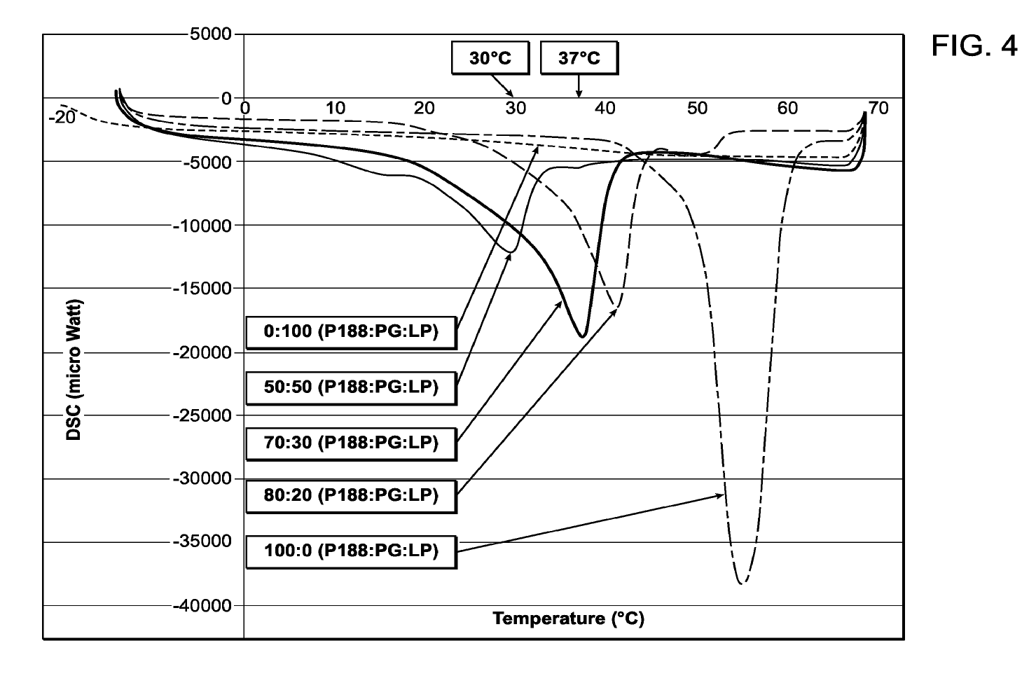

0.4 M laurocapram (17) was added to the P188/PG preparations. The DSC curves (FIG. 4) show that the formulation composed of 1 mM of the cytotoxic decoy peptide *Br1, a 70/30 ratio of P188/PG co-polymer and 0.4 M laurocapram has the desired thermo-sensitive properties for delivery of cytotoxic decoy peptides (delivery of *Br2 is not shown). This new formulation enhanced penetration of cytotoxic decoy peptides across the keratinized surface of mouse cervical mucosa, which resembles the stratum corneum of skin (data not shown).

The entire mouse cervical T-zone begins from the cervico-vaginal junction to the termination of the cervical T-zone, which is approximately one-third of the distance into one of the 2 cervical/uterine horns. To achieve topical delivery of decoy peptides into the entire mouse cervical T-zone, a solid FITC-tagged decoy peptide-containing co-polymer/enhancer rod was manually delivered into the endocervical canal and into one of the 2 cervical/uterine horns of a 7 month old K14E6 homozygous female transgenic mouse expressing the HPV type 16 E6 oncoprotein that synergizes with 1713-estradiol to induce low-grade dysplasia (CIN I, II) in situ that closely mimics the step-wise development of human cervical cancer; the “untreated” cervical served as an internal control.

After animals were anesthetized, a “speculum” comprised of a standard P-100 pipette tip with the tip cut off at the midpoint along its length was inserted into the vagina to expose the opening of the cervical canal. A three-fourth inch long, 24-guage catheter (Teruma Surflush; inner diameter 0.47 mm) with the trocar removed was fit onto a compatible P-10 micropipette. The catheter was loaded with the peptide-containing, liquid co-polymer/enhancer formulation to a length that approximates the entire cervical T-zone and extends across the squamo-columnar junction into the uterine horn.

After the co-polymer/enhancer formulation solidified into rod shape at room temperature, the tip of the catheter was inserted into the entrance of the cervix, and the solid rod was pushed into the cervical canal by the trocar; meanwhile, the catheter tip was withdrawn so that, at the end of the procedure, the rod protruded ˜2 mm outside of the cervical canal. It is observed that the co-polymer/enhancer formulation underwent phase transition from a solid rod into a consistent gel-like formulation, without leakage from the cervical opening.

Six hours after peptide delivery the animals were euthanized. The entire reproductive tract was surgically removed, fixed with formalin and embedded for histological sectioning as described.

1 mM FITC-conjugated *Br1 was used for direct visualization of peptide delivery by fluorescence microscopy. Fluorescence microscopy showed that both formulations, which contain P188 and PG, are mucoadhesive to the keratinized apical surface.

In a direct comparison between the penetration property of the lauracopram-containing and the non-lauracopram-containing formulation in a single mouse reproductive tract, a rod consisting of the 70/30 P188/PG formulation (without lauracopram) was introduced high up into the right cervical/uterine horn under ultrasound guidance, while a rod consisting of the 70/30 formulation and 0.4M lauracopram was introduced into the left cervical/uterine horn.

Specifically, penetration across the apical keratin layer of cervical squamous epithelium was only observed in the left cervical/uterine horn, where the rod contained 0.4 M lauracopram. It is evident that the lauracopram-containing rod penetrated through multiple layers of cervical squamous epithelial cells, when comparing fluorescence microscopic imaging of an unstained section with light microscopic examination of a consecutive H&E stained section.

### Example 2

### Transdermal Delivery of Insulin Glargine and Insulin Detemir in Ex Vivo 3-D Metabolically Active Full Thickness Human Skin Equivalents Using Co-Polymer/Enhancer Formulations

Proof-of-concept ex vivo studies of transdermal delivery of long-acting insulin analogues by a poloxamer 188, propylene glycol and lauracopram formulation designated TopiconDM™ were performed using metabolically active full thickness human skin equivalent (HSE) inserts from EpidermFT™ (MatTek) grown on a semi-permeable membrane that allows test compounds to pass through the tissue and accumulate in the receptor fluid on the other side. It is important to note that the term TopiconDM™ is a trademark, is not descriptive of the subject formulation, and is merely an indication of the source of the subject formulation. Thus, the use of the term TopiconDM™ herein shall not be used to affect any trademark rights associated with the term TopiconDM™.

Notably, the EpidermFT™ full thickness HSE is barrier-enhanced with a stratum corneum nearly 2-3 times that of normal human skin. EpidermFT™ tissues and other HSE models do not have structures hair follicles, sweat pores or any other structures present in normal human skin that are potential sites of transdermal delivery. All transdermal delivery in this model occurs exclusively by permeation across the barrier-enhanced stratum corneum. Drug permeation studies were modeled according to classic Franz cell in-sink diffusion studies. Studies were carried out for 5-7 continuous days to determine whether a 7-day TopiconDM™ basal insulin patch containing an insulin or insulin analogue is feasible. A semi-high throughput protocol was developed for the use of EpidermFT™, a barrier enhanced, mitotically and metabolically active human skin tissue model.

To determine the feasibility of creating a 7-day (weekly) TopiconDM™ insulin glargine patch, a protocol was developed in which the tissue inserts were incubated in tissue culture maintenance medium (with serum). All insulin and insulin analogues were commercially purchased and lyophilized. TopiconDM™ insulin glargine formulations were specifically adjusted to pH 4.0; all other insulin formulations were adjusted to pH ˜7 were prepared to deliver doses ranging from 0.01-50 IU in a volume of 50 microliters at 32° C. applied to the apical surface of individual 0.6 cm² EpidermFT™ (MatTek) full thickness human skin equivalent tissues in 6-well plates containing 2.0 ml of receptor fluid incubated at 32° C. throughout the duration of each study. It was empirically determined that 50 microliters of the formulations was sufficient to cover the entire surface of the tissue inserts when applied directly to the center. 200 microliter samples from the receptor chamber on the basal side were removed at 0.5, 1, 2, 3, 6, 8, 12, 20, 24 hours and then every 24 hours for up to 7-days; 200 microliters of fresh maintenance medium with serum was added back to receptor chamber after each sampling (closed system model). Plasma insulin and insulin analogue accumulation in mIU/cm² (reflecting transdermal delivery across 0.6 cm² tissue surface area) was quantified using the Iso-Insulin ELISA Kit (Mercodia AB, Uppsala, Sweden).

In the 0-50 IU/0.6 cm² dose range, insulin or insulin analogue steady-state flux was maintained for 5-7 days (FIG. 5-10). A similar study was conducted substituting human growth hormone for insulin in the 0.2-20 mcg dose range (FIG. 17). In the absence of P188, a key component of the Topicon™ formulation, steady-state flux was reduced by ˜50% for the 20 IU/0.6 cm² dose.

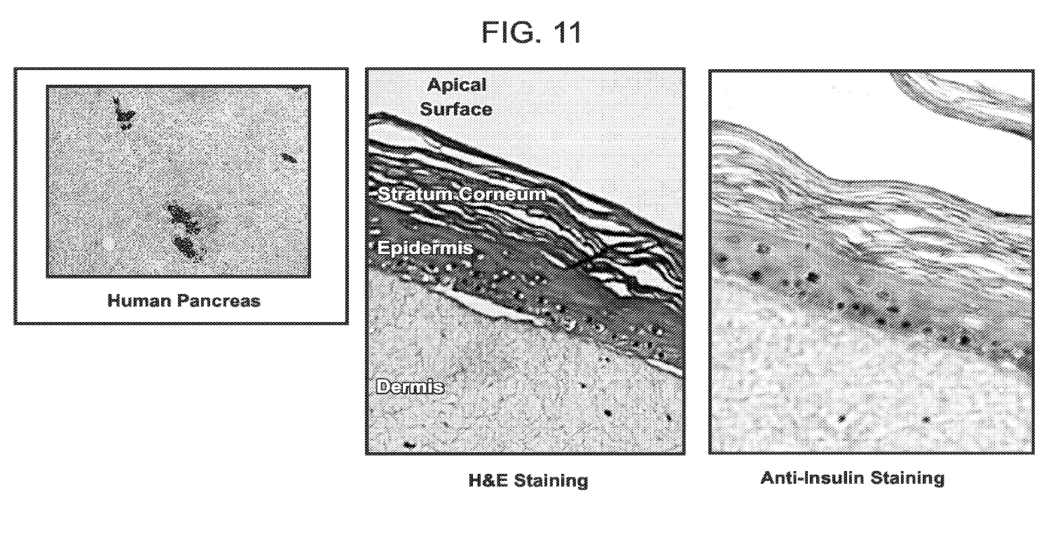

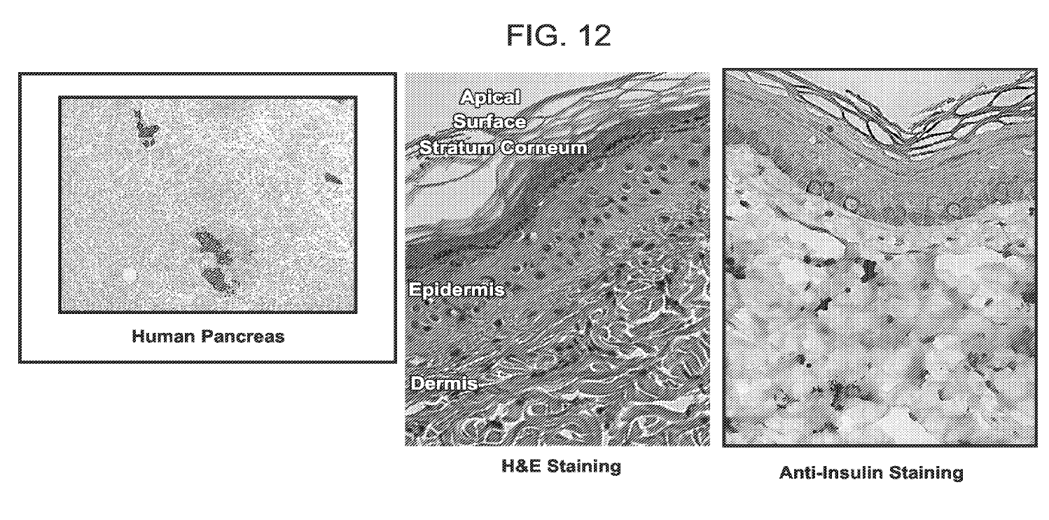

The mechanism of action of insulin glargine involves microprecipitation of monomers into tissues depots when an isoelectric shift occurs as the administered monomeric form supplied at pH 4.0 reaches physiologic tissue pH of 7.0. In FIGS. 11 and 12, immunohistochemical studies confirmed the localization of glargine microprecipitates at the dermal-epidermal junction formed after the topical application of TopiconDM™ insulin glargine to a representative EpidermFT™ tissue or hairless rat skin. The immunoperixodase staining (brown) by an antibody against insulin is predominantly localized to the epidermis just above a dense plexus of thin-walled capillaries and venules present in the dermis of human skin. Some staining of residual TopiconDM™ insulin glargine adherent to the stratum corneum is also evident. The proximity of the glargine microprecipitates to the dermal capillary and venule plexus is the likely explanation for the observed efficient delivery of insulin glargine at doses several orders of magnitude lower than required for daily bolus subcutaneous needle injections. The thicker-walled small arteries and veins of the subcutis are far less dense. The immunoperoxidase-conjugated antibody titer was optimized using normal human pancreas as the positive control as shown in the FIGS. 11 and 12 insert. The insulin-producing β-islet cells are intensely stained while there is no apparent background staining of surrounding pancreatic cells.

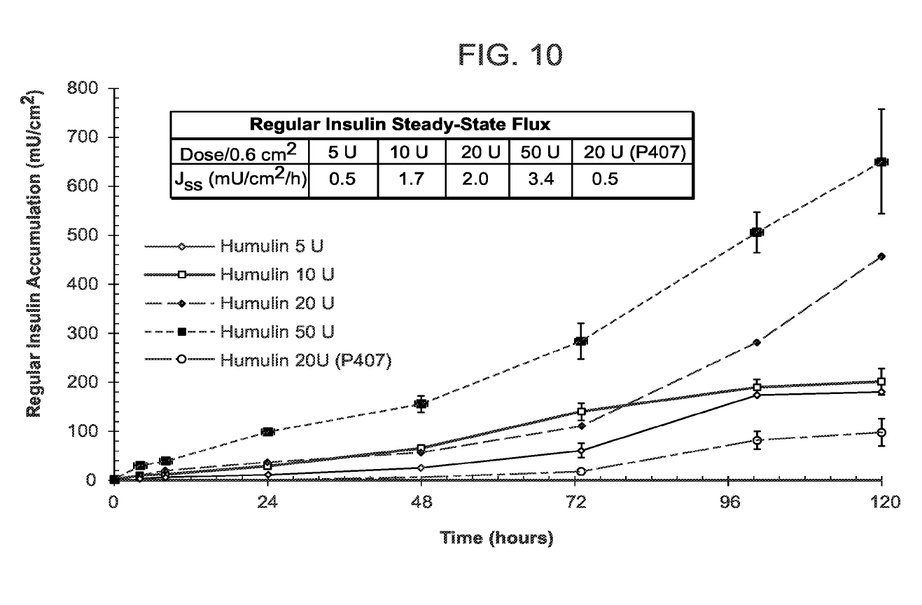

In human studies, the total plasma free insulin concentration reached a plateau concentration of 18.9 □IU/mL based on a well cited published study of glargine pharmacokinetics after a single subcutaneous injection in type 1 diabetic subjects over 3-24 hours (Lepore, M et al 2000, Diabetes 49:2142-2148). However, the actual steady-state plateau glargine concentration was 6.4 □IU/mL because the patients were titrated to an insulin concentration of ˜12.5 □IU/mL with a continuous lispro short-acting insulin infusion before glargine was administered. After scaling for average adult blood volume of 5 L and adjusting for known cross-reactivity of our ELISA assay with various insulin analogues (44% for glargine, 22% for detemir), a table comparing patch size and insulin dose required to achieve targets of 6.4 □IU/mL within 24 hours after administration of TopiconDM™ containing insulin glargine could be determined. In a 0.1-50 IU/0.6 cm² dose range the saturating dose occurs at the 10 U except for regular insulin in which no saturation was observed (FIG. 10). However, it is apparent that the 10 IU/0.6 cm² dose of insulin glargine can achieve and maintain a target steady-state total insulin concentration of 6.4 □IU/mL with a 0.6×0.6 cm patch throughout the 5-days of the study. These results suggest that a dose several orders of magnitude lower may be sufficient to achieve prolonged steady-state delivery of glargine in a patch size that would set a new industry standard for a “small patch.”

In FIG. 5 and FIG. 6, glargine or detemir accumulation was maintained for 7-days with a significant flux with a clear dose response was observed in the range of 0.01-20 IU/0.6 cm² of TopiconDM™ insulin glargine or insulin detemir, respectively, to EpidermFT™.

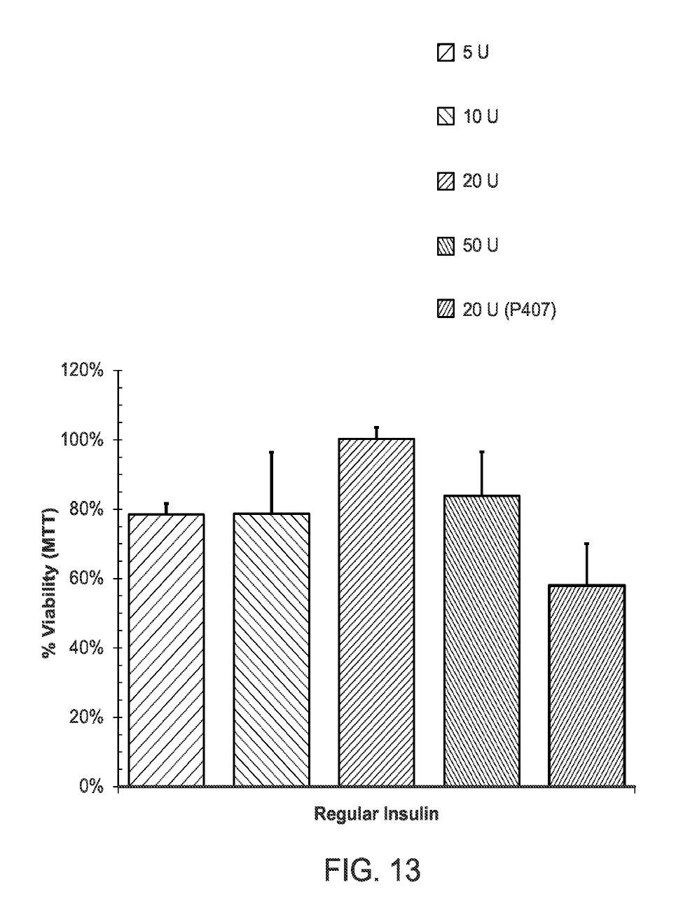

In FIG. 10 and FIG. 13, P188 was compared to P407, both poloxamers of the same class that exhibited the same thermal profile with solid to gel phase transition at ˜30° C., in order to confirm that P188 is the preferred embodiment for TopiconDM™ regular insulin, achieving higher flux and associated with epidermal cell viability. Steady-state flux decreased by 4-fold when P407 was substituted for P188. Poloxamer 188 was specifically chosen because of its thermosensitive and rheologic properties and because a purified formulation, RheothRx, had been safely administered as an intravenous preparation in randomized double-blind placebo-controlled phase III clinical trials for sickle cell disease and the setting of acute myocardial infarction. The author's suggested that it was the hemorheologic property of P188 (not thermo-sensitive property) that was disrupting hydrophobic interactions between fibrin and red blood cells. This suggested a hypothesis that P188 was not only a safe choice among all poloxamers (P407 has been reported to have renal toxicity), its rheologic properties may enhance permeation across the hydrophobic environment of the stratum corneum.

### Further Description of Example Embodiments and Data

Disclosed herein, in at least some embodiments, is a transdermal patch system that has been engineered to have thermosensitive properties, a solution to the problem of “cold chain” storage and distribution for all liquid perishable injectable drugs. The patch can be thermally profiled to maintain the stability of perishable large molecule drugs in a solid matrix at about 25° C. ambient room temperature or at any other suitable temperature. Upon patch application, the drug-containing matrix can transition into a gel state when it reaches mean skin temperature. In some embodiments, the drug-containing matrix can also act as a dermoadhesive or mucoadhesive gel that enhances transdermal drug delivery.

Referring to FIG. 4, digital Scanning calorimetry (DSC) profiles indicate that a 50:50 ratio of Poloxamer 188 (P188) to Propylene Glycol (PG) gives transition from solid to gel at ˜30° C., corresponding to the lower limit of the range of average mean skin temperature of 32° C. (range 30.7-38.6° C.) observed at steady-state conditions that included air temperatures of 20-55° C.; a 70:30 ratio gives transition from solid to gel at 37° C. core body temperature for applications such as intravaginal transmucosal delivery of chemotherapy to pre-cancerous cervical dysplasia (as detected by PAP smear). The Lauraocapram (LP) concentration is constant at 12% by weight in this figure. The ratio of P188/PG/LP is thus 44%/44%/12% (wt/wt/wt) for the formulation that has the ideal thermal properties that allow a phase transition from solid to gel at skin temperature.

Some embodiments herein were successfully tested across a full-thickness human skin model, e.g., the following compounds: Glargine (LANTUS®) 6,603 Da, Detemir (LEVEMIR®) 5,917 Da, Lispro (HUMALOG®) 5,814 Da, Glulisine (APIDRA®) 5,823 Da, Aspart (NOVOLOG®) 5,826 Da, Regular (Humulin®) 5,808 Da.

### Glargine Flux in TopiconDM™ 0.01-20 U/0.6 cm² (MatTek EpidermFT™)

Referring to FIG. 5, glargine flux was studied in a full thickness human skin model MatTek EpidermFT™ (EFT-300) that has a significantly thicker dermis than other models (e.g. StrataTest), which may make it a more physiologic model. The MatTek EpidermFT™ (EFT-300) tissues are also documented to have more of the structural features of the basement membrane. It was observed that a clear dose-response for insulin glargine in the dose range of 0.01-20 U/0.6 cm² over the course of the 6-day study. The saturating dose for insulin glargine is 10 U/0.6 cm² since the flux is not increased at a dose of 20 U/0.6 cm². Indeed, the accumulation curves for these doses are superimposable. Results reflect ELISA measurements (Mercodia Iso-Insulin) performed in triplicate for samples at each time point.

### Pharmacokinetics of Glargine Patients with Type 1 Diabetes (0.3 U/Kg Dose)

The mean plateau plasma concentration of insulin glargine is ˜6.4 micoU/mL following a single subcutaneous injection of 0.3 U/mL of insulin glargine in patients with type 1 diabetes.

TABLE 1
Achievable Plasma Insulin Glargine Concentration (mU/mL)
at 24 h (Target: 6.4 mU/mL mean plateau 24 hr after
one sc injection in patients with type 1 DM)

| Dimension | Area | | | | | |
|---|---|---|---|---|---|---|
| (cm × cm) | (cm²) | 0.01 U | 0.1 U | 1 U | 10 U | 20 U |
| 0.6 × 0.6 | 0.36 | 3.9 | 3.9 | 5.1 | 6.8 | 7.0 |
| 0.7 × 0.7 | 0.49 | 5.3 | 5.3 | 6.9 | 9.3 | 9.5 |
| 0.8 × 0.8 | 0.64 | 6.9 | 7.0 | 9.1 | 12.1 | 12.4 |

TopiconDM™ Can Achieve Target Plasma Insulin Glargine Concentration (6.4 mU/mL) Table 1 shows the combination of insulin dose vs. patch size that can achieve and maintain the target insulin glargine concentration of 6.4 microU/mL within 24 hours based on the accumulation curves in the prior figure. The concentration of insulin glargine achieved is based on accumulation in 2.5 ml of receptor volume in the tissue model extrapolated to average adult blood volume of 5 L.

In the context of these studies, a 10 U/0.6 cm² dose is predicted to achieve and maintain the target insulin glargine concentration of 6.4 microU/mL with a 0.6 cm×0.6 cm patch throughout the course of the 6-day study. Similarly, a 0.01 U/0.6 cm² dose can achieve and maintain the target insulin glargine concentration of 6.4 microU/mL with a 0.8 cm×0.8 cm patch throughout the course of the 6-day study.

Across 3-orders of magnitude dose range of insulin glargine, the size of the patch required to achieve and maintain target levels remains under 1 cm×1 cm.

### Detemir Flux in TopiconDM™ 0.01-20 U/0.6 cm² (MatTek EpidermFT™)

Referring to FIG. 6, detemir flux was studied in a full thickness human skin model MatTek EpidermFT™ (EFT-300) that has a significantly thicker dermis than other models (e.g. StrataTest), which may make it a more physiologic model. The MatTek EpidermFT™ (EFT-300) tissues are also documented to have more of the structural features of the basement membrane It was observed a clear dose-response for insulin glargine in the dose range of 0.01-20 U/0.6 cm² over the course of the 6-day study. The saturating dose for insulin glargine is 10 U/0.6 cm² since the flux is not increased at a dose of 20 U/0.6 cm². Indeed, the accumulation curves for these doses are superimposable. Results reflect ELISA measurements (Mercodia Iso-Insulin) performed in triplicate for samples at each time point.

### Insulin Detemir Pharmacokinetics (Patients with Type 1 DM)

The Cₘₐₓ for adults was 208 microU/mL after a single 0.5 U/kg dose of insulin detemir.

TABLE 2
Achievable Plasma Insulin Detemir Concentration (mU/mL)
at 6 h (Target: 208 mU/mL mean plateau 8 hr after
one sc injection in patients with type 1 DM)

| Dimension | Area | | | | | |
|---|---|---|---|---|---|---|
| (cm × cm) | (cm²) | 0.01 U | 0.1 U | 1 U | 10 U | 20 U |
| 3.0 × 3.0 | 9.0 | 68.1 | 67.9 | 83.6 | 174.4 | 207.5 |
| 3.3 × 3.3 | 10.9 | 82.4 | 82.2 | 101.2 | 211.1 | 251.0 |
| 4.8 × 4.8 | 23.0 | 174.3 | 173.8 | 214.1 | 446.5 | 531.1 |
| 5.3 × 5.3 | 28.1 | 212.5 | 211.9 | 261.0 | 544.4 | 647.5 |

TopiconDM™ Can Achieve Target Plasma Insulin Detemir Concentration (208 mU/mL) In the context of these studies, a 10 IU/0.6 cm² dose is predicted to achieve and maintain the target insulin detemir concentration of 208 microU/mL with a 3.3 cm×3.3 cm patch throughout the course of the 6-day study. Similarly, a 0.01 U/0.6 cm² dose can achieve and maintain the target insulin detemir concentration of 208 microU/mL with a 5.3 cm×5.3 cm patch throughout the course of the 6-day study.

The concentration of insulin glargine achieved is based on accumulation in 2.5 ml of receptor volume in the tissue model extrapolation to average adult blood volume of 5 L.

### Lispro Flux in TopiconDM™ 0.1-10 U/0.6 cm² (MatTek EpidermFT™)

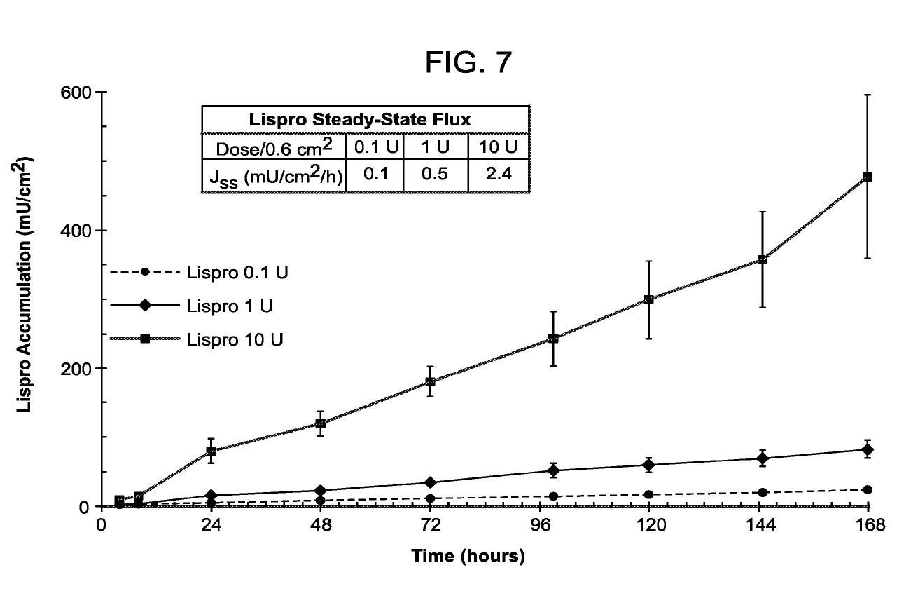

Referring to FIG. 7, insulin lispro flux was studied in a full thickness human skin model MatTek EpidermFT™ (EFT-300) that has a significantly thicker dermis than other models (e.g. StrataTest), which may make it a more physiologic model. The MatTek EpidermFT™ (EFT-300) tissues are also documented to have more of the structural features of the basement membrane.

In this preliminary study, a clear dose-response was observed for insulin lispro in the dose range of 0.1-10 U/0.6 cm² over the course of the 7-day study. Results reflect ELISA measurements (Mercodia Iso-Insulin) performed in triplicate for samples at each time point.

### Glulisine Flux in TopiconDM™ 0.1-10 U/0.6 cm² (MatTek EpidermFT™)

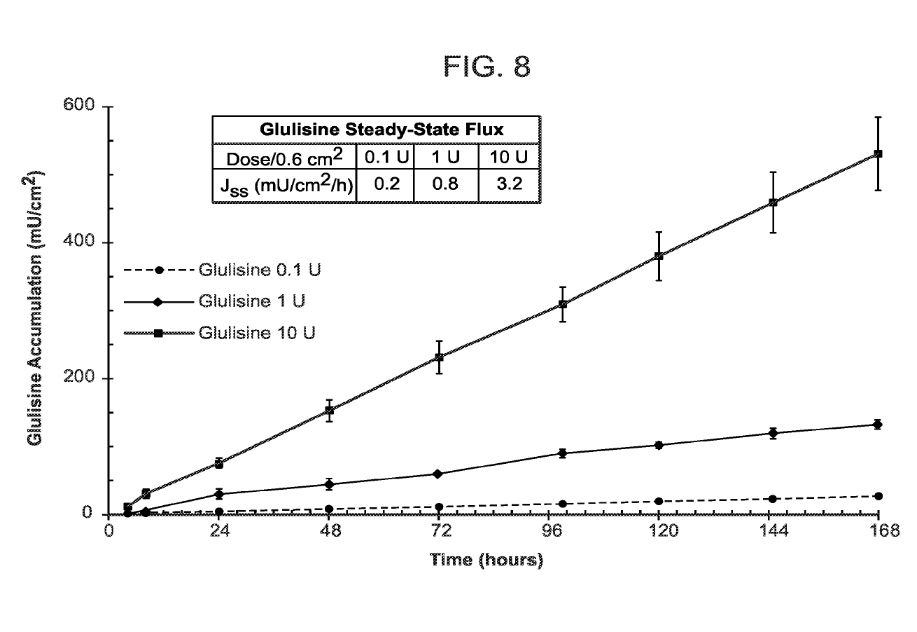

Referring to FIG. 8, insulin glulisine flux was studied in a full thickness human skin model MatTek EpidermFT™ (EFT-300) that has a significantly thicker dermis than other models (e.g. StrataTest), which may make it a more physiologic model. The MatTek EpidermFT™ (EFT-300) tissues are also documented to have more of the structural features of the basement membrane.

In this preliminary study, a clear dose-response was observed for insulin glulisine in the dose range of 0.1-10 U/0.6 cm² over the course of the 7-day study. Results reflect ELISA measurements (Mercodia Iso-Insulin) performed in triplicate for samples at each time point.

### Aspart Flux in TopiconDM™ 0.1-10 U/0.6 cm² (MatTek EpidermFT™)

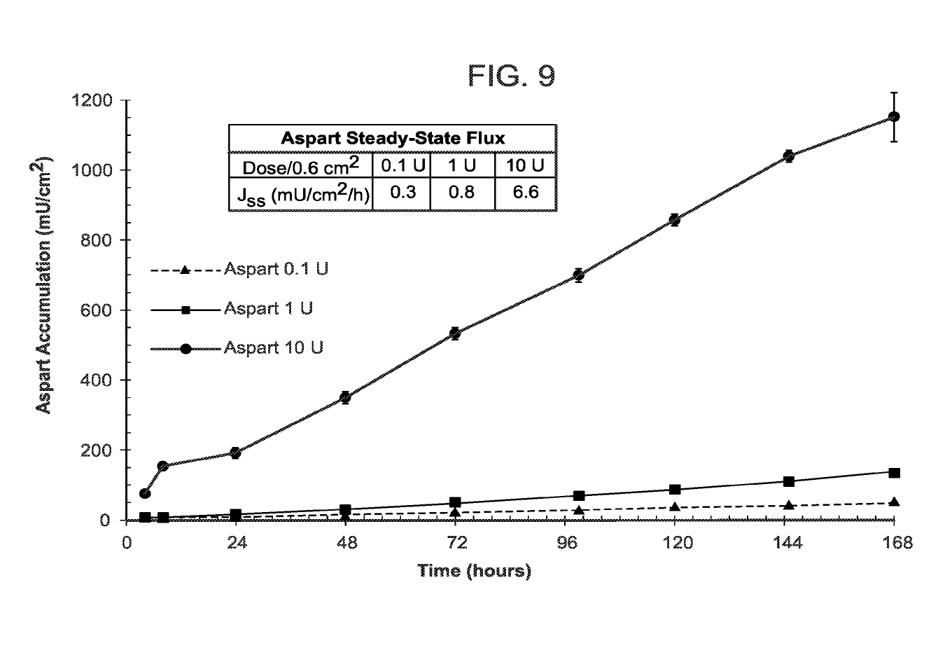

Referring to FIG. 9, insulin aspart flux was studied in a full thickness human skin model MatTek EpidermFT™ (EFT-300) that has a significantly thicker dermis than other models (e.g. StrataTest), which may make it a more physiologic model. The MatTek EpidermFT™ (EFT-300) tissues are also documented to have more of the structural features of the basement membrane.

In this preliminary study, a clear dose-response was observed for insulin aspart in the dose range of 0.1-10 U/0.6 cm² over the course of the 7-day study. Results reflect ELISA measurements (Mercodia Iso-Insulin) performed in triplicate for samples at each time point.

### Role of Poloxamer 188 in TopiconDM™ Comparison of P188 vs P407

Referring to Table 3, Poloxamers are nonionic triblock copolymers composed of a central hydrophobic chain of polyoxypropylene [poly(propylene oxide)] flanked by two hydrophilic chains of polyoxyethylene [poly(ethylene oxide)]. Some poloxamers are also known by the trade names Synperonics, Pluronics and Kolliphor. P188 vs. P407, the two most commonly used poloxamers in human applications, were compared. P407 has been reported to have kidney toxicity. P188 has been used in a purified form and administered by continuous intravenous infusion in the context of sickle cell crisis based on the hypothesis that its rheologic properties may normalize capillary blood flow by altering red cell morphology and disrupting interactions between cell transmembrane proteins and the endothelium. A definitive randomized, double-blind, placebo-controlled Phase III study demonstrated statistically beneficial effects on all pre-specified primary endpoints on patients hospitalized with sickle cell crisis. However, the effect size was apparently not large enough to justify the continued development of therapy with intravenous purified P188 for this clinical indication. GSK discontinued development towards commercialization. Notably, intravenous infusion of purified P188 was shown to be safe in this study.

TABLE 3

| | Physical | Average Molecular | Weight % | Unsaturation, |
|---|---|---|---|---|
| Poloxamer | Form | Weight | Oxyethylene | mEq/g |
| 124 | Liquid | 2090 to 2360 | 46.7 ± 1.9 | 0.020 ± 0.008 |
| 188 | Solid | 7680 to 9510 | 81.8 ± 1.9 | 0.026 ± 0.008 |
| 237 | Solid | 6840 to 8830 | 72.4 ± 1.9 | 0.034 ± 0.008 |
| 338 | Solid | 12700 to 17400 | 83.1 ± 1.7 | 0.031 ± 0.008 |
| 407 | Solid | 9840 to 14600 | 73.2 ± 1.7 | 0.048 ± 0.017 |

### Regular Insulin Flux in TopiconDM™ 0-50 U/0.6 cm² (MatTek EpidermFT™) and Regular Insulin Steady State Flux: P188 vs. P407

Referring to FIG. 10, regular insulin flux was studied in a full thickness human skin model MatTek EpidermFT™ (EFT-300) that has a significantly thicker dermis than other models (e.g. StrataTest), which may make it a more physiologic model. The MatTek EpidermFT™ (EFT-300) tissues are also documented to have more of the structural features of the basement membrane.

In this preliminary study, a clear dose-response was observed for regular insulin in the dose range of 10-50 U/0.6 cm² over the course of the 5-day study. Unlike the case with insulins glargine and detemir, no saturation was observed at the highest dose. Results reflect ELISA measurements (Mercodia Iso-Insulin) performed in triplicate for samples at each time point.

Poloxamer 188 was specifically selected for its rheologic properties from among hundreds of classes of thermosensitive compounds and from among the entire class of poloxamers (tri-block polymers). Substitution of Poloxamer 407 for Poloxamer 188, while maintaining transition from solid to gel at about 30° C., is associated with a dramatic decrease in flux at the non-saturating dose of regular insulin of 20 U/0.6 cm². Results reflect ELISA measurements (Mercodia Iso-Insulin) performed in triplicate for samples at each time point.

### Glargine Microprecipitates Form in Epidermis of EpidermFT™ within 4-Hours (1 U/0.6 cm² Insulin Glargine in TopiconDM™)

Referring to FIG. 11, it was confirmed that the localization at the dermal-epidermal junction of glargine microprecipitates formed after the topical application of TopiconDM™ insulin glargine to a representative EpidermFT™ tissue. The immunoperixodase staining (brown) by an antibody against insulin is predominantly localized to the epidermis just above a dense plexus of thin-walled capillaries and venules present in the dermis of human skin. Diffuse light and diffuse staining is also seen in the semi-permeable membrane, representing insulin glargine monomers crossing this membrane to enter the receptor fluid on the basal side. Some staining of residual TopiconDM™ insulin glargine adherent to the stratum corneum is also evident. The proximity of the glargine microprecipitates to the dermal capillary and venule plexus is the likely explanation for the observed efficient delivery of insulin glargine at doses several orders of magnitude lower than required for daily bolus subcutaneous needle injections. The thicker-walled small arteries and veins of the subcutis are far less dense. The immunoperoxidase-conjugated antibody titer was optimized using normal human pancreas as the positive control as shown in the insert. The insulin-producing beta-islet cells are intensely stained while there is no apparent background staining of surrounding pancreatic cells.

### Glargine Microprecipitates Form in Epidermis of Rat Skin within 4-Hours (5 U/1 cm² Insulin Glargine in TopiconDM™)

Referring to FIG. 12, it was confirmed that the localization at the dermal-epidermal junction of glargine microprecipitates in a presentative hairless rat, formed at the site of topical application of a TopiconDM™ patch containing glargine 5 U/1 cm² applied for 4-hours followed by full thickness skin biopsy. The immunoperixodase staining (brown) by an antibody against insulin is predominantly localized to the epidermis just above a dense plexus of thin-walled capillaries and venules present in the dermis of human skin. The proximity of the glargine microprecipitates to the dermal capillary and venule plexus is the likely explanation for the observed efficient delivery of insulin glargine at doses several orders of magnitude lower than required for daily bolus subcutaneous needle injections. The thicker-walled small arteries and veins of the subcutis are far less dense. The immunoperoxidase-conjugated antibody titer was optimized using normal human pancreas as the positive control as shown in the insert. The insulin-producing beta-islet cells are intensely stained while there is no apparent background staining of surrounding pancreatic cells.

### MTT Assay of Cell Viability Dose-Response and Comparison of P188 vs. P407

Referring to FIG. 13, Cell viability of MatTek EpidermFT™ (EFT-300) tissues, as assessed by the standard MTT assay, is not significantly reduced (80-100%) after exposure to a TopiconDM™ patch formulation containing regular insulin over the dose range of 5-50 U/1 cm²) for 7-days. Each bar represents the mean±SD of tissues treated in triplicate. The values represent percentage of cell viability compared to untreated control tissues (set at 100%). Substitution of Poloxamer 407 for Poloxamer 188, while maintaining transition from solid to gel at 30° C., is associated with a decrease in cell viability of 60% at the non-saturating regular insulin dose of 20 U/0.6 cm² at the end of this 5-day study. This is consistent with previous reports of cell toxicity and adverse effects on the kidney. Thus, P407 is associated with both a 4-fold decrease in flux and 60% cell viability compared to P188 as a component of the TopiconDM™ patch formulation.

### Insulin Glargine PK/PD-SC Injection (STZ-Induced Male CD Hairless Rat: Type 1 DM)

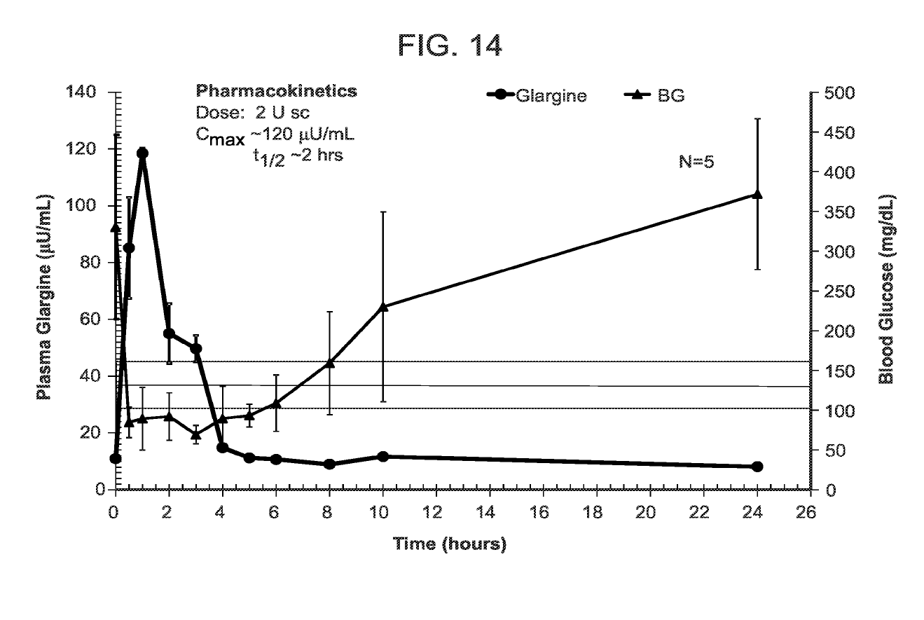

Referring to FIG. 14, shown is a 24 Hour PK/PD study showing rapid appearance in and elimination of SC Injected Glargine from the Systemic Circulation. The red plot is the glargine concentration from pooled plasma (average±std dev of ELISA measurements). Blue plot is blood glucose (BG) concentration (average±std dev) measured by ACCU-CHEK® Aviva glucose meter. Average BG (blue line) and range (white horizontal bar) in normal CD hairless rats was determined to be ˜130 mg/dL (100-160 mg/dL) in a cohort of adult rats weighing ˜250 gm.

PK (Y-axis scale on left): A single insulin glargine 2 U subcutaneous injection in adult rats (˜250 gm) resulted in rapid rise in plasma concentration and rapid elimination as evidenced by Cₘₐₓ˜120 microU/mL attained within 1 hour, a short t_{1/2} (˜2 hours) and return to baseline level by 5 hours.

PD (Y-axis scale on right): The blood glucose (BG) and plasma glargine plots are essentially minor images of each other, consistent with an expected dramatic BG lowering effect after insulin glargine injection. The baseline mean BG of ˜375 mg/dL rapidly reached a nadir of ˜70-80 mg/dL that was persistent between 0.5-5 hours, representing asymptomatic hypoglycemia.

Rats that received 2 U sc injection rapidly became hypoglycemic (defined as BG<100 mg/dL), but were not symptomatic because they were observed to increase food consumption when BG dropped. All subsequent PK studies of insulin glargine subcutaneous administration were performed with a dose of 1 U, which is the smallest dose that can be injected accurately without dilution of commercially purchased LANTUS® (U-100). The manufacturer warns against diluting the insulin glargine.

ELISA to quantitate the plasma glargine level was performed in triplicate on pooled blood for each time point (alternating N=2 and N=3) using the Mercodia Iso-Insulin kit. Blood samples were pooled to allow for adequate sampling of many time points over the course of 24-hour study and to reduce variability. This method has been validated for performing PK studies.

TopiconDM™ Needleless™ Patch Delivery can Achieve Insulin Glargine Bioavailability and Induce a Therapeutic Response Comparable to SC Injection Glargine PK/PD: TopiconDM™ Needleless™ Patch (STZ-Induced Male CD Hairless Rat: Type 1 DM)

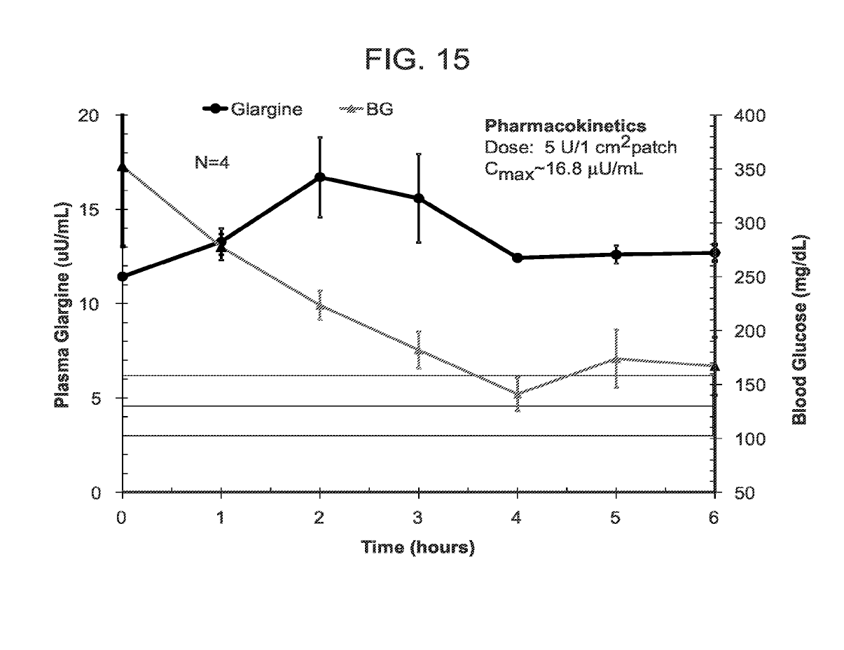

Referring to FIG. 15, average BG (blue line) and range (white horizontal bar) in normal CD hairless rats was determined to be ˜130 mg/dL (100-160 mg/dL) in a cohort of adult rats weighing ˜250 gm.

Green plot is plasma glargine concentration (average±std dev). Brown plot is blood glucose concentration (average±std dev) measured by ACCU-CHEK® Aviva glucose meter. Measurements were obtained from all individual animals (N=4) at each time point (i.e. blood samples were not pooled). ELISA to quantitate the plasma glargine level was performed in triplicate on blood samples from individual animals (N=4) at each time point using the Mercodia Iso-Insulin kit.

The plasma glargine and blood glucose (BG) plots are essentially mirror images of each other, consistent with BG lowering effect into the euglycemic range within 4 hours. Notably, the plasma glargine concentration achieves a Cₘₐₓ at 2 hours and maintains a steady-state plateau between 4-6 hours associated with a BG level in the euglycemic range between 4-6 hours.

### Glargine PD: TopiconDM™ Patch Vs. Needle (STZ-Induced Male CD Hairless Rat: Type 1 DM)

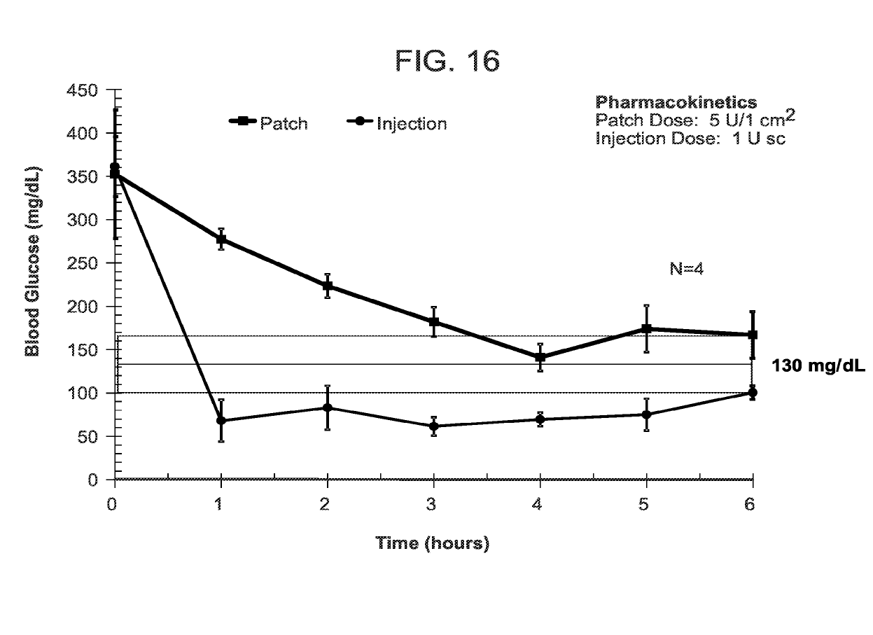

Referring to FIG. 16, A PD study of insulin glargine BG lowering effect comparing 1 U subcutaneous (sc) injection (gold standard) versus 5 U/1 cm² patch was performed. Referring to FIG. 18, the blue plot is BG concentration (average±std dev) of rats that received a single 1 U subcutaneous injection. Brown plot is BG concentration of rats that received a 5 U/1 cm² patch. BG measurements by ACCU-CHEK® Aviva glucose meter were obtained from all individual animals (N=4) at each time point (i.e. blood samples were not pooled). ELISA to quantitate the plasma glargine level was performed in triplicate on blood samples from individual animals (N=4) at each time point using the Mercodia Iso-Insulin kit.

Average BG (blue line) and range (white horizontal bar) in normal CD hairless rats was determined to be ˜130 mg/dL (100-160 mg/dL) in a cohort of adult rats weighing ˜250 gm. Rats that received 1 U sc injection rapidly became hypoglycemic (BG<100 mg/dL), but were not symptomatic because they were observed to increase food consumption when BG dropped. Delivery of a smaller dose of insulin glargine by injection was not feasible because there are no syringes that can accurately measure a smaller volume than 1 U (10 microliters) of undiluted commercially purchased LANTUS® (U-100). It was observed from a prior dose-response experiment that a 5 U/1 cm² patch was the optimum patch dose capable of achieving BG lowering into the normal range within 4 hours and maintaining this euglycemic state for the remainder of this 6 hour study.

### hGH Flux in TopiconDM™ 0.2-20 microgram/0.6 cm² (EpiDermFT®)

Referring to FIG. 17, Human growth hormone (hGH) flux is shown over a dose range of 0.2-20 ug/0.6 cm² and was observed to have a clear dose-response over a 7-day study.

### EQUIVALENTS

The functions of several elements may, in alternative embodiments, be carried out by fewer elements, or a single element. Similarly, in some embodiments, any functional element may perform fewer, or different, operations than those described with respect to the illustrated embodiment. Also, functional elements (e.g., modules, databases, computers, clients, servers and the like) shown as distinct for purposes of illustration may be incorporated within other functional elements, separated in different hardware, or distributed in a particular implementation.

While certain embodiments according to this disclosure have been described, this disclosure is not limited to just the described embodiments. Various changes and/or modifications can be made to any of the described embodiments without departing from the spirit or scope of this disclosure. Also, various combinations of elements, steps, features, and/or aspects of the described embodiments are possible and contemplated even if such combinations are not expressly identified herein.

### INCORPORATION BY REFERENCE

The entire contents of all patents, published patent applications, and other references cited herein are hereby expressly incorporated herein in their entireties by reference.

### APPENDIX

### Non-Limiting Embodiments of Pharmaceuticals for Incorporation into Transdermal Patches

| Trademark(s) | Generic Name | Compan(ies) | Disease/Medical Use |
|---|---|---|---|
| NEXIUM | Esomeprazole | AstraZeneca | Gastrointestinal |
| | | | disorders |
| ABILIFY | Aripiprazole | Otsuka Pharmaceutical, | Psychosis, Depression |
| | | Bristol-Myers Squibb | |
| CRESTOR | Rosuvastatin | AstraZeneca, Shionogi | Cholesterol |
| CYMBALTA | Duloxetine | Eli Lilly and Company | Depression, Anxiety |
| | | | disorders |
| HUMIRA | Adalimumab | Abbott Laboratories | Rheumatoid arthritis |
| ADVAIR DISKUS, | Fluticasone + | GlaxoSmithKline | Asthma |
| SERETIDE | Salmeterol | | |
| ENBREL | Etanercept | Amgen | Rheumatoid arthritis |
| REMICADE | Infliximab | Johnson & Johnson, | Crohn's disease, |
| | | Schering-Plough, | Rheumatoid arthritis |
| | | Mitsubishi Tanabe | |
| | | Pharma | |
| COPAXONE | Glatiramer | Teva Pharmaceutical | Multiple sclerosis |
| | | Industries, Sanofi- | |
| | | Aventis | |
| NEULASTA | Filgrastim | Amgen | Neutropenia |
| RITUXAN, | Rituximab | Roche, Genentech, | Non-Hodgkin's |
| MABTHERA | | Biogen Idec, Chugai | lymphoma, |
| | | Pharmaceutical | Rheumatoid arthritis |
| ATRIPLA | Emtricitabine/tenofovir/ | Gilead Sciences, Inc. | HIV infection |
| | efavirenz | | |
| LIPITOR | Atorvastatin | Pfizer | Cholesterol |
| SPIRIVA | Tiotropium | Boehringer Ingelheim | Chronic obstructive |
| | | | pulmonary disease |
| JANUVIA | sitagliptin | Merck & Co., Inc. | Cholesterol |
| AVASTIN | Bevacizumab | Roche, Genentech | Colorectal cancer |
| LANTUS | Insulin analog | Sanofi-Aventis | Type 2 diabetes and |
| | | | type 1 diabetes |
| TRUVADA | Tenofovir + | Gilead Sciences | HIV infection |
| | Emtricitabine | | |
| LANTUS SOLOSTAR | Insulin analog | Sanofi-Aventis | Type 2 diabetes and |
| | | | type 1 diabetes |
| EPOGEN | Erythropoietin | Amgen | Anemia |
| LYRICA | Pregabalin | Pfizer | Neuropathic pain |
| CELEBREX | Celecoxib | Pfizer | Osteoarthritis and |
| | | | rheumatoid arthritis |
| SINGULAIR | Montelukast | Merck & Co. | Asthma |
| DIOVAN | Valsartan | Novartis | Hypertension |
| GLEEVEC, GLIVEC | Imatinib | Novartis | Leukemia |
| HERCEPTIN | Trastuzumab | Roche, Genentech, | Breast cancer |
| | | Chugai Pharmaceutical | |
| NAMENDA | Memantine | Forest Laboratories | Alzheimer disease |
| SUBOXONE | Buprenorphine | Reckitt Benckiser | Acute Withdrawal |
| | | Pharmaceuticals Inc. | Symptoms |
| AVONEX | Interferon beta-1a | Biogen Idec | Multiple sclerosis |
| ENOXAPARIN | Enoxaparin | Generic | Deep-vein thrombosis |
| LUCENTIS | Ranibizumab | Genentech, Inc | Anemia |
| ESCITALOPRAM | Escitalopram | Generic, H. Lundbeck | Depression, Anxiety |
| | | | disorders |
| DIOVAN HCT | Valsartan | Novartis | Hypertension |
| VYVANSE | Lisdexamfetamine | Shire US Inc | Attention Deficit |
| | | | Disorder/Sleepiness |
| ZETIA | Ezetimibe | Merck & Co., | Cholesterol |
| | | Schering-Plough | |
| ANDROGEL | Ezetimibe | Abbott Laboratories, | Cholesterol |
| | | Schering-Plough | |
| METHYLPHENIDATE | Methylphenidate | Generic | Attention-deficit |
| | | | hyperactivity disorder |
| ACTOS | Pioglitazone | Takeda | Type 2 diabetes |
| | | Pharmaceutical, Eli | |
| | | Lilly and Company | |
| LIDODERM | Lidocaine | Endo Pharmaceuticals | Pain |
| TRICOR, LIPANTHYL | Fenofibrate | Abbott Laboratories, | Cholesterol |
| | | Solvay | |
| REBIF | Interferon beta-1a | Serono | Multiple sclerosis |
| SYMBICORT | Budesonide + | AstraZeneca | Asthma |
| | Formoterol | | |
| NOVOLOG | Insulin aspart + | Novo Nordisk Inc. | Chronic obstructive |
| | Salbutamol | | pulmonary disease |
| INCIVEK | Erythropoietin | Johnson & Johnson | Anemia |
| SEROQUEL XR | Quetiapine | AstraZeneca | Schizophrenia, Bipolar |
| | | | Disorder |
| ALIMTA | Pemetrexed | Eli Lilly and Company | Non-small cell lung |
| | | | cancer |
| VIAGRA | Sildenafil | Pfizer | Erectile dysfunction |
| LEVEMIR, INSULIN | Insulin detemir | Novo Nordisk Inc. | Anemia |
| DETEMIR | | | |
| AMPHETAMINE/ | amphetamine/dextroamphetamine | Generic | Attention Deficit |
| DEXTROAMPHETAMINE | | | DIsorder/Sleepiness |
| NOVOLOG FLEXPEN | Insulin aspart + | Novo Nordisk Inc. | Chronic obstructive |
| | Salbutamol | | pulmonary disease |
| PROAIR HFA | Insulin aspart + | Teva Pharmaceuticals | Chronic obstructive |
| | Salbutamol | | pulmonary disease |
| LOVAZA | Darbepoetin alfa | GlaxoSmithKline | lower very high |
| | | | triglyceride |
| COMBIVENT | Ipratropium + | Boehringer Ingelheim | Chronic obstructive |
| | Salbutamol | | pulmonary disease |
| VARIVAX, | Oka/Merck strain of | Merck & Co., Inc. | Anemia |
| OKA/MERCK STRAIN | live | | |
| OF LIVE | | | |
| NIASPAN | Niacin | Kos Pharmaceuticals | Cholesterol |
| PROCRIT, EPREX | Erythropoietin | Johnson & Johnson | Anemia |
| HUMALOG | Insulin analog | Eli Lilly and Company | Diabetes |
| METOPROLOL, | Metoprolol | Generic | Hypertension |
| METOPROLOL | | | |
| NEUPOGEN | Filgrastim | Amgen | Neutropenia |
| NASONEX | Mometasone | Schering-Plough | Allergic rhinitis |
| FLOVENT HFA | Fluticasone | GlaxoSmithKline | Asthma |
| REYATAZ | Atazanavir | Bristol-Myers Squibb | HIV infection |
| ISENTRESS | Raltegravir | Merck & Co., Inc. | HIV Infection |
| VYTORIN | Ezetimibe + | Merck & Co., | Cholesterol |
| | Simvastatin | Schering-Plough | |
| MODAFINIL | Modafinil | Generic | Sleepiness |
| JANUMET | metformin and | Merck & Co., Inc. | type 2 diabetes |
| | sitagliptin | | |
| CIALIS | Tadalafil | Eli Lilly and Company, | Erectile dysfunction |
| | | Lilly Icos | |
| ACETAMINOPHEN/ | acetaminophen/hydrocodone + | Generic | Asthma/analgesia |
| HYDROCODONE | Formoterol | | |
| GILENYA | vaccine | Novartis Corporation | Pneumococcal disease |
| ARANESP | Insulin detemir | Amgen Inc. | Anemia |
| RESTASIS | cyclosporine | Allergan, Inc, Lilly | Chronic Dry Eye and |
| | ophthalmic emulsion | Icos | Inflammation |
| ACIPHEX, PARIET | Rabeprazole | Eisai, Johnson & | Gastrointestinal |
| | | Johnson | disorders |
| VICTOZA | Levothyroxine | Novo Nordisk Inc. | Hypothyroidism |
| PRADAXA | Tadalafil | Boehringer Ingelheim | Erectile dysfunction |
| | | Pharmaceuticals, Inc, | |
| | | Lilly Icos | |
| ORENCIA | Budesonide + | Bristol-Myers Squibb | Asthma |
| | Formoterol | Company | |
| BETASERON, | Interferon beta-1b | Schering AG | Multiple sclerosis |
| BETAFERON | | | |
| ELOXATIN, | Oxaliplatin | Sanofi-Aventis | Colorectal cancer |
| ELOXATINE | | | |
| VESICARE | vaccine | Astellas Pharma US | Pneumococcal disease |
| DEXILANT | Eszopiclone | Takeda | Insomnia |
| | | Pharmaceuticals North | |
| | | America, Inc | |
| ADDERALL XR | Amphetamine | Shire Pharmaceuticals | Attention-deficit |
| | | | hyperactivity disorder |
| FENTANYL | Fentanyl | Generic | Pain |
| PREZISTA | Rabeprazole | Eisai, Johnson & | Gastrointestinal |
| | | Johnson | disorders |
| ZOSTAVAX | Zostavax | Eisai, Merck & Co. | live vaccine |
| EVISTA | Raloxifene | Eli Lilly and Company, | Osteoporosis |
| | | Chugai Pharmaceutical | |
| BENICAR, OLMETEC | Olmesartan | Daiichi Sankyo, Forest | Hypertension |
| | | Laboratories | |
| GARDASIL, SILGARD | Olmesartan | Merck & Co., Forest | human papillomavirus |
| | | Laboratories | |
| LIPITOR | Atorvastatin | Pfizer | Cholesterol |
| BUDESONIDE | Budesonide + | Generic | Asthma |
| | budesonide | | |
| SYNTHROID | Levothyroxine | Abbott Laboratories | Hypothyroidism |
| XOLAIR | Omalizumab | — | Hypothyroidism |
| RENVELA | Olmesartan | Genzyme Corporation, | Hypertension |
| | | Forest Laboratories | |
| LUNESTA | Eszopiclone | Sepracor | Insomnia |
| PREVNAR 13 | vaccine | Wyeth | Pneumococcal disease |
| XELODA | Capecitabine | Roche, Chugai | Cancer |
| | | Pharmaceutical | |
| EPIPEN 2-PAK | EpiPen 2-Pak | Dey Pharma | Bacterial infections |
| SENSIPAR | Sensipar | Amgen Inc. | Bacterial infections |
| ZYVOX | Linezolid | Pfizer | Bacterial infections |
| ATORVASTATIN | Atorvastatin | Generic | Cholesterol |
| STELARA | Ustekinumab | Pfizer | inflammatory disorders |
| ARANESP | Darbepoetin alfa | Amgen | Anemia |
| ARANESP | Darbepoetin alfa | Merck & Co., Inc. | Anemia |
| VELCADE | Tadalafil | Takeda | Erythema nodosum |
| | | Pharmaceuticals North | leprosum |
| | | America, Inc, Lilly | |
| | | Icos | |
| PRADAXA | Tadalafil | Merck & Co., Inc., | Erectile dysfunction |
| | | Lilly Icos | |
| ERBITUX | Cetuximab | Bristol-Myers Squibb, | Colorectal cancer |
| | | Merck KGaA | |
| VENTOLIN HFA | Levothyroxine | GlaxoSmithKline | Hypothyroidism |
| PROTONIX, | Pantoprazole | Wyeth, Altana, Solvay | Gastrointestinal |
| PANTOZOL, | | | disorders |
| PANTOLOC | | | |
| TAMIFLU | Oseltamivir | Roche, Chugai | Influenza |
| | | Pharmaceutical, Hetero | |
| | | Drugs | |

1. A device for measurement and monitoring of a subject simultaneously with transdermal or transmucosal delivery of a therapeutic agent at a contact site with the subject's skin, the device comprising:
a transdermal sensor adapted and configured to detect a specific indicator that is either the therapeutic agent itself or a biomarker that is affected by the therapeutic agent; and
a therapeutic-agent-containing formulation for passive or active transdermal drug delivery, wherein the formulation includes a dermoadhesive agent adapted and configured to hold the sensor and the storage reservoir to the skin at the contact site.

2. The device of claim 1, wherein the therapeutic agent is selected from the group consisting of: small molecule drugs, large molecule drugs, therapeutic peptides, DNA, and microRNA.

3. The device of claim 2, wherein the therapeutic agent is selected from the group consisting of: human native insulin, insulin analogues, and insulin glargine.

4. The device of claim 1, wherein the transdermal sensor comprises one or more electrodes adapted and configured for insertion into the subepidermal interstitial space.

5. The device of claim 1, wherein the transdermal sensor is disposable.

6. The device of claim 1, wherein the transdermal sensor is an enzymatic electrochemical sensor.

7. The device of claim 1, wherein the transdermal sensor includes one or more electrodes adapted to pierce the skin.

8. The device of claim 7, wherein immobilized glucose oxidase is coupled at least one of the electrodes.

9. The device of claim 8, wherein:
the immobilized glucode oxidase catalyzes oxidation of glucose to hydrogen peroxide and D-glucono-6-lactone; and
the hydrogen peroxide reacts with a surface of the electrode to produce electric current.

10. The device of claim 4, wherein the specific indicator is an interstitial glucose level that is correlated with a blood glucose level of the subject.

11. (canceled)

12. (canceled)

13. The device of claim 1, wherein a surface of the device adjacent to the contact site is between about 4 and about 25 square centimeters in size.

14. The device of claim 1, wherein the device is between about 4 and about 25 cubic centimeters in size.

15. (canceled)

16. The device of claim 1, wherein the dermoadhesive agent is selected from the group consisting of propylene glycol, dipropylene glycol, polyethylene glycol, glycerine, butylene glycol, glycol derivatives with glycerol esters, and non-ionizable glycol ether derivatives.

17. The device of claim 1, wherein the storage reservoir is a transdermal skin patch.

18. The device of claim 1, wherein the therapeutic-agent-containing formulation includes a thermo-sensitive polymer.

19. The device of claim 18, wherein thermo-sensitive polymer is a poloxamer or a poloxamine.

20. The device of claim 19, wherein the poloxamer is poloxamer 188.

21. The device of claim 1, wherein the storage reservoir releases the therapeutic agent via passive or active diffusion across a skin surface that is not being physically disrupted with technologies selected from the group consisting of: thermal or laser ablation, microneedles, high pressure jet injection, ultrasound, sonar and iontophoresis.

22.-31. (canceled)

32. A method of monitoring a subject comprising:
applying a device of claim 1 to the skin of a subject.

33. The method of claim 32, wherein the device is maintained in place on the skin of the subject for at least 1 day.

34. (canceled)

35. (canceled)

36. The method of claim 32, further comprising:
transmitting instructions to modulate a dosage of the therapeutic agent to the subject based on the specific indicator detected by the transdermal sensor.

37. The method of claim 32, wherein the storage reservoir releases the therapeutic agent via passive or active diffusion across a skin surface that has not been physical disrupted with technologies selected from the group consisting of: thermal or laser ablation, microneedles, high pressure jet injection, ultrasound, sonar and iontophoresis.

38. A method of making the device of claim 1, comprising: attaching the transdermal sensor system to the contact site with a dermoadhesive agent.

39. (canceled)

40. (canceled)

41. (canceled)

42. (canceled)

43. (canceled)

44. (canceled)

45. (canceled)
